(19) United States
(12) Patent Application Publication
(10) Pub. No.: US 2022/0305405 A1
(43) Pub. Date: Sep. 29, 2022

(54) SYSTEM AND METHOD FOR ISOLATION OF INTACT EXTRACELLULAR VESICLES WITH NEAR-SINGLE-VESICLE RESOLUTION COUPLED WITH ON-LINE CHARACTERIZATION

(71) Applicant: The Research Foundation for The State University of New York, Albany, NY (US)

(72) Inventors: Chioma OKEOMA, Stony Brook, NY (US); Hussein SADDOUR, Middle Island, NY (US)

(73) Assignee: The Research Foundation for The State University of New York, Albany, NY (US)

(21) Appl. No.: 17/607,960

(22) PCT Filed: May 1, 2020
(86) PCT No.: PCT/US2020/030914

§ 371 Date: Nov. 1, 2021

Related U.S. Application Data
(60) Provisional application No. 62/841,448, filed on May 1, 2019.

(51) Int. Cl. B01D 15/34; G01N 30/82; G01N 30/46; B01D 15/18

(52) U.S. Cl. CPC B01D 15/34; G01N 30/82; G01N 30/466; B01D 15/1871; G01N 2030/8813

(57) ABSTRACT

A method and system are disclosed for isolating intact acellular particles using size exclusion and for obtaining size and concentration of such isolated particles. In one embodiment, the disclosure is directed to use of Particle Purification Liquid Chromatography (PPLC), a high-resolution chromatographic size-guided turbidimetry-enabled system for dye-free isolation, on-line characterization, and retrieval of intact acellular particles, including extracellular vesicles (EVs) and membraneless condensate particles (MCs) from various biofluids.

This application claims priority under 35 U.S.C. § 119 to provisional application U.S. Ser. No. 62/841,448, filed May 1, 2019, the entire contents of which are incorporated herein by reference.

### GOVERNMENT SUPPORT STATEMENT

This invention was made with government support under DA042348 awarded by the National Institutes of Health. The government has certain rights in the invention.

### FIELD

The disclosure pertains to purification and characterization, including size and concentration, of intact acellular particles such as extracellular vesicles and membraneless condensate particles.

### BACKGROUND

Most mammalian cells produce and release small acellular particles or structures into biofluids, including cell culture medium, urine, saliva, milk, blood, and semen. These particles perform divergent physiological and pathophysiological functions depending on the cellular background that released them. Two archetypes of acellular particle architectures are present in biofluids. One type is characterized by the presence of lipid bilayer membrane and a second type is characterized by the absence of a membrane. However, both archetypes have the presence of bioactive cargo in common; i.e., assemblage of proteins and nucleic acids.

One of the most widely studied lipid bilayer membrane-encased particle are the nano-sized extracellular vesicles (EVs) such as exosomes, small −30-150 nm vesicles secreted by most cell types. These EVs, membrane-enclosed nanoparticles, facilitate distal and proximal intercellular communications and are present in all body fluids, such as cerebrospinal fluid, urine, blood, saliva, breast milk, vaginal fluid, and semen. Seminal studies in the past decade have demonstrated that EVs largely orchestrate recipient cells' fate by inducing pathogenesis, promoting tumor progression, and regulating neurodegenerative disorders, among other roles. The diversity of EV-mediated regulations of cellular function has been attributed to (i) its bioactive cargo, including of mRNA, miRNA, proteins, lipids, and dsDNA, and (ii) ability to protect the cargo against degradation. These properties of EVs as well as their endogenous nature allowed them to be considered as promising candidates of drug delivery and therapeutic agents.

The other acellular particle archetype are the membraneless particles or membraneless condensates (MCs) that concentrate a wide array of bioactive molecules without an encapsulating membrane. According to in vitro studies, MCs assemble by thermodynamic-mediated liquid-liquid phase separation (LLPS) and they Aggregate biomolecules in concentrations. MCs have been shown to regulate biological process, including but not limited to RNA metabolism, chromatin rearrangement, and signal transduction. Noteworthy is that liquid MCs can transform into solid aggregates or reversible amyloid fibers. The amyloid fibers have been linked to the pathogenesis of amyotrophic lateral sclerosis), frontotemporal dementia and even Alzheimer's disease. Thus, MCs may be biologically important in many processes.

The isolation of EVs and MCs from biofluids requires stringent control to ensure quality and production of particles that meet advanced analytical characterization preparative needs. Meeting these requires instrumentation with comprehensive in-line monitoring and retrieval capability, both of which will facilitate control of critical process parameters, such as particle purity, stratification into sub-populations, and retrieval of preparative quantities. However, research on and use of EVs and MCs have been largely hindered by technical difficulties related to the aforementioned parameters.

Currently, there are numerous methods available to purify EVs from body fluids and tissue culture samples, but none for MCs These EV methods include differential ultracentrifugation, ultrafiltration, density gradient, flow cytometry, immunocapture, microfluidic isolation, SEC, and Asymmetric Flow Field Flow Fractionation (AF4). None of these methods has been demonstrated efficient in sub-population isolation that allows downstream functional analysis. Recently, AF4 was significantly optimized permitting the identification of membraneless EVs, coined “exomeres”. Though interesting, this technology has limitations, and requires some level of special skills and expensive instrumentation. As a result, AF4 is not broadly accessible.

At present, EV properties, especially exosome concentration and size, are determined using the Nanoparticle Tracking Analysis (NTA) technique. Briefly, NTA is a light scattering-based method that measures the Brownian motion of a particle. The speed of motion or diffusion constant is related to the size of the particle that can be calculated using the Stockes-Einstein equation. The NTA system includes a camera that captures scattered light from each particle which is tracked independently over multiple frames, thus allowing, to determine the particle concentration using mathematical derivation. However, the NTA system is very expensive and has limitations. First, NTA (and any light scattering technique) assumes that EV are spherical, which is not true. Indeed, it was proven by cryo-TEM imaging that EVs derived from a single cell type ex vivo have diverse shapes and sizes, much more so than EVs derived from body fluids in vivo. Second, NTA requires very dilute samples (1:40,000-1:100,000) raising questions about precision of measurements and reproducibility. NTA also has a high background noise. Indeed, measurements of filtered saline would give a typical size distribution and a concentration of −10⁵ particles/ml, indicative of room for errors in data interpretation. Furthermore, NTA cannot achieve in-line determination of size and concentration because: (1) NTA employs brownian motion which is affected by the velocity generated by the flow during separation skewing size determination; (2) NTA requires very dilute samples whereas the separation would generate variable concentration of vesicles across the chromatogram; and (3) NTA require CCD/cMOS camera, a very expensive option, that is not needed for Ultraviolet-visible spectroscopy (UV-Vis or UV-VIS) based concentration determination of particles. And while UV-Vis has been used to determine the concentration of EVs, the wavelength used was 280 nm where proteins absorb light. Thus UV-Vis at 280 nm cannot discriminate between EV and EV-free proteins.

There is thus a need for a system and method that eliminates the variations that result from the current preparative and analytical sample preparations for EVs and MCs.

### SUMMARY

In one aspect the disclosure is directed to a method for isolating intact acellular particles, such as e.g. extracellular vesicles, membraneless condensate particles, or both. In one embodiment, the method comprises (i) providing a biofluid sample containing intact acellular particles of different sizes; (ii) separating the biofluid sample using a size exclusion gradient, such as e.g. by using a particle purification liquid chromatography column that comprises layers of size exclusion beads having different pore sizes, into subpopulations of intact acellular particles wherein each respective subpopulation individually comprises a different size range of intact acellular particles; and (iii) isolating a respective subpopulation, e.g. by elution of a respective subpopulation from the particle purification column into a fraction collector where each fraction contains a respective subpopulation of respectively different particle sizes. In another aspect, the method further comprises (iv) analyzing the respective subpopulation to determine size of the intact acellular particles therein, the concentration of the intact acellular particles therein, or both. In one practice, analyzing comprises obtaining light scattering information for a respective isolated subpopulation, such as e.g. by UV-VIS spectrometry, and using that light scattering information to determine the size and the concentration of the intact acellular particles contained in that respective subpopulation. In another aspect, a distinct RNA profile, a distinct DNA profile, and/or a distinct proteome for each respective subpopulation can be obtained from the method, as well as quantification of total lipids in a respective subpopulation. The method may be practiced dye-free, thus avoiding associated complications.

### BRIEF DESCRIPTION OF THE DRAWINGS

FIG. 1AA is an absorbance spectra of the output of an embodiment of an SEC column using the method of the disclosure showing registration of lipid content, particle size, and particle concentration in the visible range (400 nm-600 nm).

FIG. 1A depicts an elution of EVs through monosized beads.

FIG. 1B depicts an elution of EVs through an embodiment of gradient sized beads as in the disclosure.

FIG. 1C depicts a flow scheme for clarification of a crude EV mixture.

FIG. 1D graphically depicts elution of clarified seminal plasma through monosized bead columns G-10, G-100, and an embodiment of gradient sized bead column as in the disclosure.

FIG. 1E graphically depicts elution of clarified seminal plasma through an embodiment of gradient sized bead columns of different lengths.

FIG. 1F graphically depicts seminal plasma separation profiles for four different donors using an embodiment of a gradient sized bead column of the disclosure.

FIG. 1G graphically depicts blood plasma and serum separation profiles for four different donors using an embodiment of a gradient sized bead column of the disclosure.

FIG. 1H graphically depicts cow milk (whole, 2%, fat free) separation profiles using an embodiment of a gradient sized bead column of the disclosure.

FIG. 1I graphically depicts a clarified and concentrated urine sample separation profile from a donor using an embodiment of a gradient sized bead column of the disclosure.

FIG. 1J graphically depicts separation profiles for various cell cultures using an embodiment of a gradient sized bead column of the disclosure.

FIG. 2A graphically depicts separation profiles for four collected fractions F1-F4 for seminal plasma after using an embodiment of a gradient sized bead column of the disclosure.

FIG. 2B is a representative picture of the four collected fractions of FIG. 2A after volume adjustment.

FIG. 2C graphically depicts the size and concentration of vesicles in the four collected fractions of FIG. 2A using NTA.

FIG. 2D graphically depicts the zeta potential of vesicles in the four collected fractions of FIG. 2A using NTA.

FIG. 2E graphically depicts the AChE enzymatic activity of vesicles in the four collected fractions of FIG. 2A.

FIG. 2F graphically depicts total protein of vesicles in the four collected fractions of FIG. 2A.

FIG. 2G is a representative SDS-PAGE profile showing the protein profile of vesicles in the four collected fractions of FIG. 2A.

FIG. 2H is a representative Western Blot analysis of known EV markers in the four collected fractions F1-F4 of FIG. 2A.

FIG. 2I is a representative negative stain TEM imaging of the four fractions F1-F4 of FIG. 2A.

FIG. 2J is a representative TEM-based mean particle size determination for the vesicles in the four fractions F1-F4 of FIG. 2A.

FIG. 3A is a representative UV-VIS spectra of the four fractions F1-F4 of FIG. 2A.

FIG. 3B is a representative contour view of 3D UV spectra of the four fractions F1-F4 of FIG. 2A.

FIG. 3C is a representative 3D surface plot of the four fractions F1-F4 of FIG. 2A in the turbidity range.

FIG. 3D is a representative comparison of turbidity (middle, A₄₀₀, A₆₀₀, A₆₅₀) as against A₂₈₀ (top) of the four fractions F1-F4 of FIG. 2A. Bottom graph depicts the corresponding profiles of R1 (A₄₀₀/A₆₀₀) and R2 (A₆₀₀/A₆₅₀) ratios.

FIGS. 4A to 4C respectively graphically depict separation profiles showing absorbance at 280 nm, NP fluorescence, and turbidity profiles of a seminal sample separated by an embodiment of a size exclusion column of the disclosure.

FIG. 4D depicts R₁ and R₂ ratios.

FIG. 4E graphically depicts a representative standard curve for turbidity and NP fluorescence of POPC vesicles fit to a simple linear regression function.

FIGS. 4F to 4G graphically depict a representative total lipid concentration per fraction calculated using the equations of the fluorescence (FIG. 4F) and turbidity (FIG. 4G) linear fit calculated in FIG. 4E.

FIG. 4H depicts a representative non-linear regression between the total lipid concentrations as determined by NP fluorescence and turbidity of FIGS. 4F and 4G, respectively.

FIG. 5A is a visual representation showing that the same total lipid concentration in a solution can form different combinations of heterogeneous size and particle concentration.

FIGS. 5B and 5C are respectively representative modeled spectra for hypothetical hollow spheres with varying lipid concentration at a fixed particle size of 100 nm, and particle size at a fixed lipid concentration of 1 mM.

FIG. 5D graphically depicts representative separation profiles showing 280 nm absorbance, where the inset represents the 3D contour view of an F1 area, indicated by a gray lane.

FIG. 5E is a representative turbidity spectra of the F1 area of FIG. 5D.

FIG. 5F graphically depicts a representative EV particle concentration and hydrodynamic diameter (Dₕ) of FIG. 5D as calculated from the measured turbidity spectra.

FIG. 5G graphically depicts a representative particle size and concentration of individual fractions determined by NTA of FIG. 5D.

FIG. 5H and FIG. 5I are respectively linear regression for size and particle concentration between the turbidity model and NTA data of FIGS. 5G and 5E.

FIG. 6A shows RNA Bioanalyzer profiles of DNase I-treated RNA isolated from four fractions F1-F4 that were separated from human seminal plasma using an embodiment of the size exclusion column of the disclosure.

FIG. 6B shows denaturing PAGE results from fractions F1-F4 of FIG. 6A, untreated or treated with DNase I, or with RNase cocktail (RNase A+RNase T1).

FIG. 7A is a cluster heatmap of the various seminal plasma proteins (2178) identified by the method of the disclosure in this study.

FIG. 7B is a Venn diagram of the common and distinct proteins found in representative fractions for F1-F3 as determined by the spectral count (SpC) method.

FIG. 7C is a Venn diagram showing the common and distinct proteins significantly enriched in one fraction as compared to the other two fractions as obtained by the method of the disclosure.

FIG. 7D is a heatmap of the differentially enriched protein in each fraction F1, F2, F3 as obtained by the method of the disclosure.

FIG. 7E illustrates flow diagrams for non-redundant GO Terms of the differentially enriched proteins in each fraction as determined by Webgestalt analysis.

FIGS. 7F and 7G respectively show AUC of seven previously known and cell-free transcription factors identified by the method of the disclosure, as quantified in each of the three fractions F1, F2, F3.

FIG. 8A is a graphical depiction of a separation profile using an embodiment of the method of the disclosure for U1 cells infected with HIV, the inset showing twelve fractions.

FIG. 8B shows results of a Bradford and ACHE activity analyses of the twelve fractions from FIG. 8A.

FIG. 8C is a Western blot analysis of EV markers and viral proteins for the twelve fractions of FIG. 8A.

FIG. 8D is a graph showing quantification of the bands in FIG. 8C. Gray bar depicts fractions with HIV particles.

FIG. 8E is a 3D UV-Vis profile of the separation of FIG. 8A, with the turbidity range enlarged at the bottom and showing 2 distinct subpopulations of particles.

FIG. 9A is a graphical depiction of a separation profile using an embodiment of the method of the disclosure of human seminal plasma being tested for anti-HIV function and showing three fractions chosen for analysis.

FIG. 9B is a depiction of the three collected fractions of FIG. 9A adjusted to the same volume.

FIG. 9C is a schematic flow for a method of the disclosure for an HIV infection assay.

FIG. 9D is a graphical representation showing the effect of HIV infection on Tzm-bl with SEV_{L}, SEV_{S} and MC treatment.

FIG. 9E is a graphical representation showing Tat-mediated HIV promoter activation in Tzm-bl with SEV_{L}, SEV_{S} and MC treatment.

FIG. 9 F is a Venn diagram between HIV-interactome and the proteins identified in Example 1 (FIG. 7) showing 459 potential anti-HIV proteins.

FIG. 10A is absorbance spectra at 280 nm for clarified blood plasma after being purified by the method of the disclosure using an SEC column.

FIG. 10B is Bradford analysis of the fractions identified in FIG. 10A.

FIG. 10C is an SDS-PAGE analysis of the fractions identified in FIG. 10A.

FIG. 10D is a Western Blot analysis of EV markers and albumin in the fractions of FIG. 10A.

FIG. 10E shows ImageJ quantification of the bands in FIG. 10D. Gray bar depicts EV fractions.

FIG. 10F shows turbidity ratio R₁ of the separation profile related to fractions identified in FIG. 10A.

FIG. 10G is 3D-UV-Vis profile for fractions identified in FIG. 10A, with a focus on the turbidity region.

FIG. 10H is a contour view of the UV region for fractions identified in FIG. 10A.

### DETAILED DESCRIPTION

The following detailed description of embodiments of the disclosure are made in reference to the accompanying figures. Explanation about related functions or constructions known in the art are omitted for the sake of clearness in understanding the concept of the invention to avoid obscuring the invention with unnecessary detail. Embodiments of the disclosure described herein provide a system and method for isolation of intact acellular particles such as Extracellular Vesicles (EV) with near-single-vesicle resolution coupled with on-line characterization. The system and method facilitates disease-specific biomarker discoveries as well as development of new strategies for treatment of currently uncurable diseases.

In one embodiment, the disclosure is directed to use of Particle Purification Liquid Chromatography (PPLC), a high-resolution chromatographic size-guided turbidimetry-enabled system for dye-free isolation, on-line characterization, and retrieval of intact acellular particles, including extracellular vesicles (EVs) and membraneless condensate particles (MCs) from various biofluids. In one practice, chromatographic separation of acellular particles from biofluids derived from various cell cultures, blood, milk, and semen, is achieved using a gradient-bead size exclusion (SEC) column. Purified intact acellular particles are then collected as sub-populations using an automated fraction collector and purification profiles obtained by ultraviolet-visible spectroscopy (UV-Vis). The UV-Vis analyses reveal sample-dependent differences in UV-Vis spectra, with milk and semen having the most complex UV-Vis spectra. Application of industry-ready turbidimetry facilitates accurate physical characterization of seminal particles (Sps), including particle lipid content, size, and concentration. Particle turbidimetry parameters can be validated against nano-tracking analysis and transmission electron microscopy. Furthermore, the naphthopyrene assay—a fluorescence-based technique that allows naphthopyrene fluorescence upon embedment into a hydrophobic environment can be used to validate detection of Sps containing lipid bilayer. Assessment of compositional content of Sps show that different fractions of purified Sps contain distinct DNA, RNA species, and protein cargos. Proteomic data can be analyzed to determine different protein compartmentalization with varied gene ontology functional predictions. Integration of Sps physical characteristics and cargo composition can be used to determine the presence of two archetypal membrane-encase large SEV (SE_{L}) and small SEV (SEV_{S}), as well as novel non-archetypal-membraneless seminal particles, classifiable as membraneless condensate particles (MCs).

In one embodiment the disclosure is directed to a method for isolating intact acellular particles which, without limitation, comprise extracellular vesicles (EVs), membraneless condensate particles (MCs), or both. The method comprises providing a biofluid sample containing intact acellular particles of different sizes. Without limitation, the biofluid sample comprises, which also includes being derived from, one or more of the following: a biological fluid, a body fluid, a culture fluid obtained from a human cell, a culture fluid obtained from a bacterial cell, a culture fluid obtained from a fungus cell. The biofluid sample is then separated using a size exclusion gradient into subpopulations of intact acellular particles, each respective subpopulation individually comprising a different size range of intact acellular particles. In one practice, the separation step comprises contacting the biofluid sample with size exclusion beads under conditions effective to separate the intact acellular particles into the subpopulations. Without limitation, the size exclusion beads comprise different pore sizes and are configured to form the gradient going from largest pore size to smallest pore size and the biofluid sample progressively contacts the gradient from largest pore size to the smallest pore size. As used herein, the term “isolating” and its variants refers to enriching the amount of intact acellular particles in the respective subpopulation to permit one or more of the ensuing analyses as described herein to occur on that subpopulation.

In one non-limiting practice, the biofluid sample is fed to the inlet of a particle purification liquid chromatography column comprising size exclusion beads having different pore sizes. The size exclusion beads are layered within the column to provide a gradient along the length of the column wherein the largest pore size is at the inlet of the column and the smallest pore size is at the outlet the column. Size exclusion beads as, e.g., known in the art for, among other things, exclusion chromatography can by utilized, including such beads comprised of cross-linked dextran gel. For example, macroscopic beads synthetically derived from the polysaccharide dextran are serviceable, including such beads wherein the organic chains are cross-linked to give a three-dimensional network having functional ionic groups attached by ether linkages to glucose units of the polysaccharide chains. Such beads can separate molecules by molecular weight. In exclusion chromatography, the fractionation range of such beads is typically given for globular proteins and Dextrans (Da). Such ranges for Dextrans for a respective gel type size exclusion bead includes the following: beads having a fractionation range of ≤700 Da; ≤1500 Da; 100-5000 Da; 500-10000 Da; 1000-50000 Da; 1000-100000 Da. Beads of this type are commercially available under the name Sephadex, e.g. Sephadex Gel Types G-10 (≤700 Da); G-15 (≤1500 Da); G-25 (100-5000 Da), including G-25 fine (1000-5000 Da); G-50 (500-10000 Da), including G-50 medium (1000-30000 Da); G-75 (1000-50000 Da, including the range of 3000-8000 Da); and G-100 (1000-100000 Da), including the range of 4000-15000 Da). Other, including but not limited to lower and higher dextran ranges, different SEC bead types, or Ion Exchange beads, may be employed. In one non-limiting practice, for a particle purification liquid chromatography column of given length, the size exclusion beads are layered from bottom to top in the following percentages: G-10 (bottom) at about 3 to 7% of column length, e.g. 5%; then G-15 at about 5 to about 9% of column length, e.g. 7.5%; then G-25 fine at about 9 to about 13% of column length, e.g. 11%; then G-75 at about 20 to about 28% of column length, e.g. 24%; and finally G-100 (top) at about 40 to about 45% of column length, e.g. 35%. In another embodiment, a hybrid Ion Exchange and gradient size exclusion beads is used.

The biofluid sample flows through the column from the inlet to the outlet under conditions effective to progressively elute respective subpopulations of intact acellular particles, where each respective subpopulation individually comprises a different size range of intact acellular particles.

The respective eluting subpopulations are isolated, e.g. by collecting the respective subpopulation of intact acellular particles as a fraction of the biofluid using a fraction collector as known in the art which typically has a plurality of wells.

In another embodiment, fraction collection of intact acellular particles, e.g. EVs, permits downstream applications such as functional studies, RNA sequencing and Matrix-Assisted Laser Desorption/Ionization (MALDI) mass spectrometry. In one practice, a fast fraction collector that is able to collect as low as 50 μl per fraction in a 96-well plate format is used; in another practice, 10 μl fractions in a 384 well plate are collected, e.g., by controlling the flow rate. A CO₂ and temperature-controlled fraction collector can be used where fractions are titrated onto pre-incubated cells in a 384 well plates. In one embodiment, the fraction collector accommodates four plates or more in series without stopping the separation. Alternatively, the fraction collector can directly spot EV fractions into an RNA sequencing plate or on a MALDI plate (with automatic pre-mixing with a chosen matrix). The latter (a MALDI-ready format fraction collector) is commercially available from Shimadzu (AccuSpot model). In another embodiment, a diagnostic tool that uses EV UV 3D profiles to indicate a physiological and/or pathological state of a patient, as well as monitor patient's response to treatment can be used. Distinct 2D and 3D UV profiles of blood and semen EVs with a new class of EV subpopulation are shown to be present in seminal plasma and absent in blood plasma. The embodiments of the disclosure provide a fully automated EVpurification system that can: (1) isolate EV to the near-single vesicle resolution; (2) accurately determine size and concentration of EV in real time; (3) allow intact EV fraction collection for downstream functional and analytical studies; and (4) be used in a clinical setting as a miniaturized device that can monitor EV profiles in patients, as a marker of disease or response to therapy.

In addition, the method comprises analyzing the respective subpopulation of intact acellular particles, e.g. the respective eluate subpopulation isolated by a fraction collector, to determine the size of the intact acellular particles therein, the concentration of the intact acellular particles therein, or both. In one embodiment, such analyzing comprises obtaining light scattering information for the respective subpopulation, which light scattering information is used to determine the size and the concentration of the intact acellular particles contained in the respective subpopulation. In one aspect, the light scattering information includes absorbance by the respective subpopulation of light in the visible range of between about 400 nm to about 600 nm. In one aspect, UV-VIS spectrometry is performed on the respective subpopulation within the one or more wells of the fraction collector to obtain light scattering information for that respective subpopulation and determining from the light scattering information the size of the intact acellular particles contained in that respective subpopulation, the concentration of the intact acellular particles contained in that respective subpopulation, or both. In one practice, a portion of the visible range (about 400 nm to about 600 nm), where turbidity derived from the presence of lipid membranes, is measured. Lipid-membrane-derived turbidity is indicative of the presence of EVs. To precisely identify lipid-membrane-derived turbidity from contaminant scatterers, turbidity ratios, R₁=A₄₀₀/A₆₀₀ and R₂=A₆₀₀/A₆₅₀, are defined as an EV existence index. In fractions where R₁ and R₂ proportionally increase above background, it is indication of the presence of EVs. When R₁ and R₂ vary disproportionally, it is indication of the presence of contaminant scatterers, such as colored materials (phenol red, bilubrin, urobilin, etc.). These turbidity ratios operate concurrently to delimit vesicle-containing fractions without the need for a PDA detector. These ratios can be interpreted as purity indicators for vesicles, in the same manner A₂₆₀/A₂₃₀ and A₂₆₀/A₂₈₀ are used in the art for nucleic acid purity assessment.

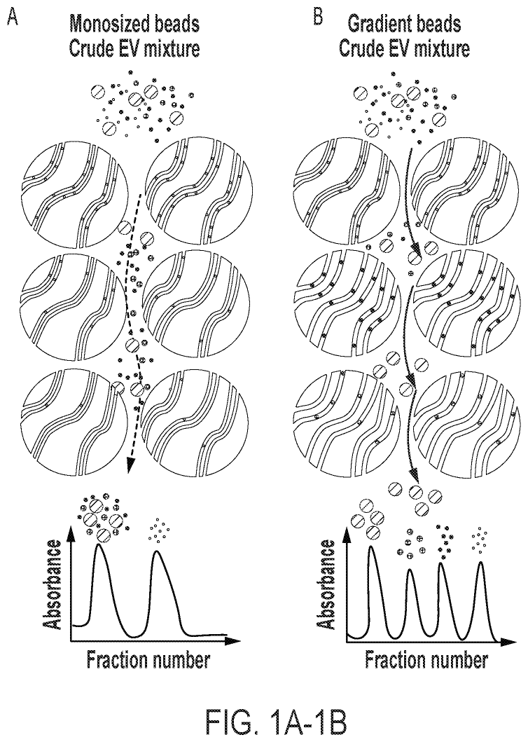

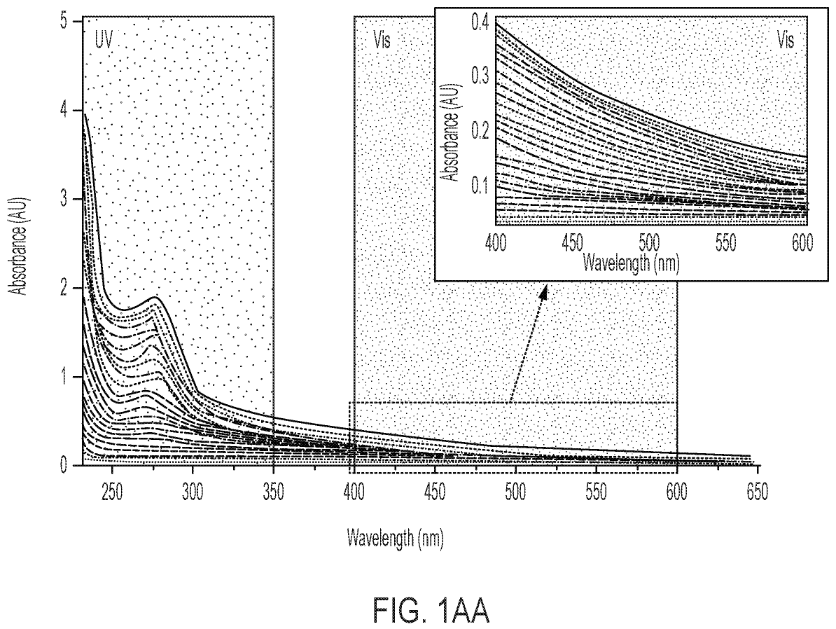

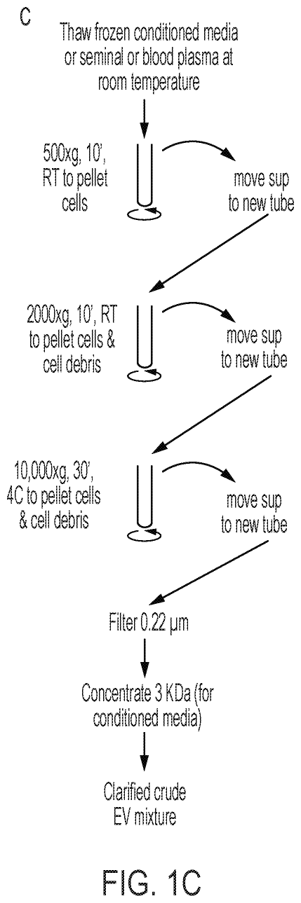

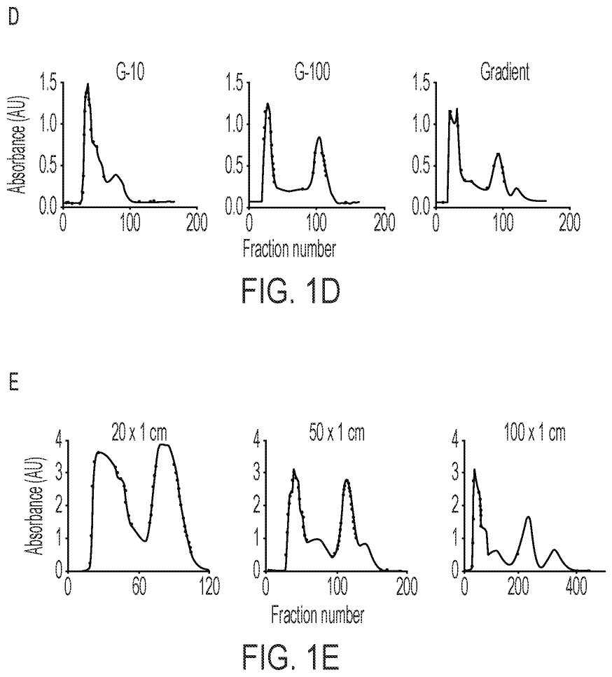

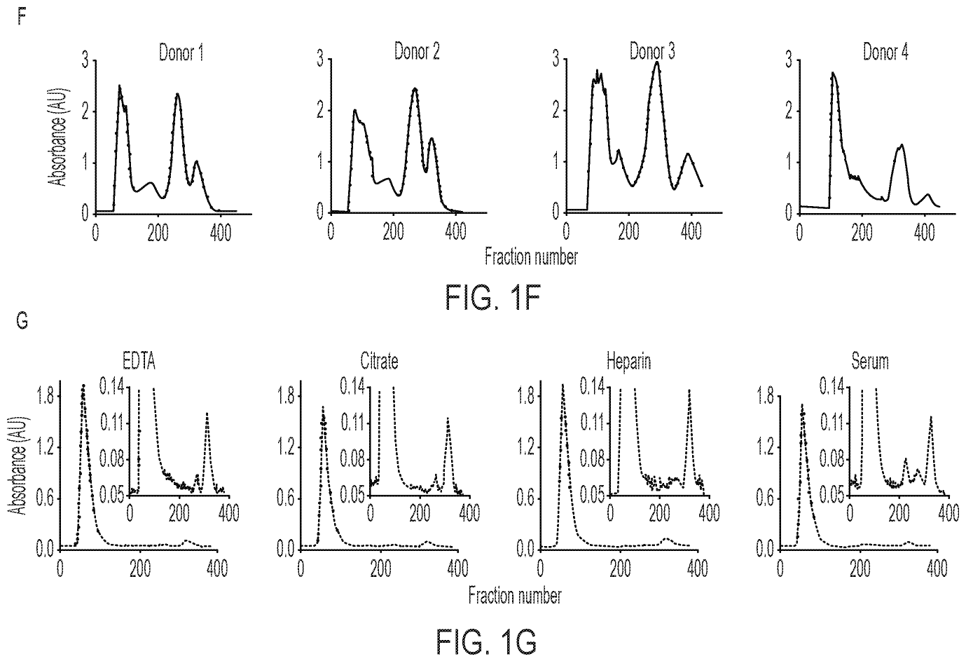

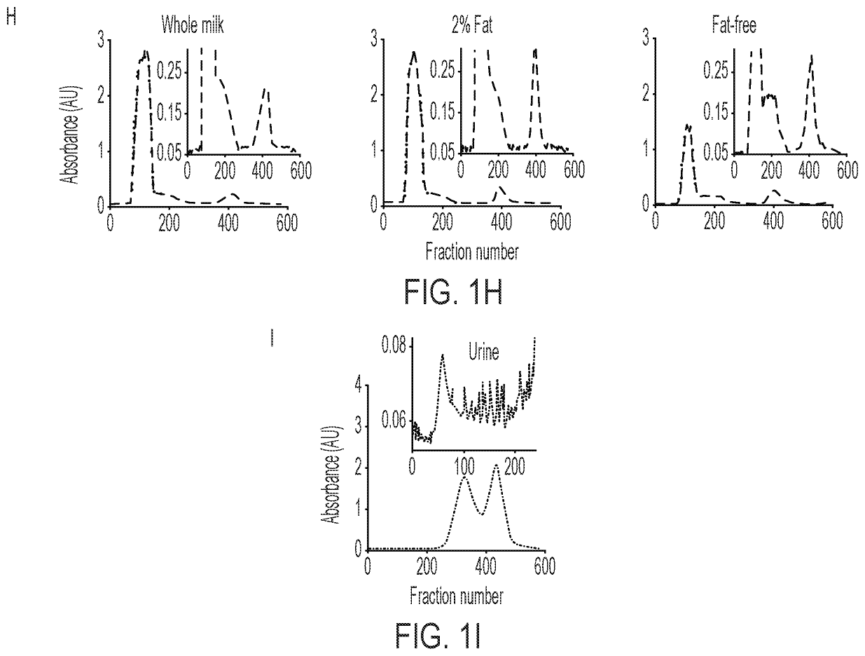

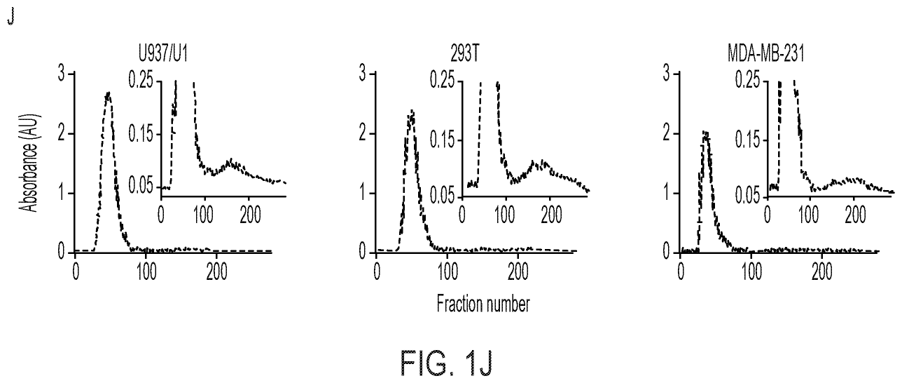

In one practice, UV-Vis spectroscopy is employed. In the UV range (230 nm-350 nm), the biomolecular fingerprint of the collected fractions, including the identity of biological cargo, concentration and purity, is extracted. In the visible range (400 nm-600 nm), information about the lipid content, particle size, and particle concentration is registered as shown in FIG. 1AA, which shows an example of PPLC output absorbance spectra. Each region of the spectra, the UV and the visible, contain information regarding the content and physical characteristics of the sample. Inset is zoom-in of the visible region. In one practice, the analysis proceeds as follows as shown in the Example hereinbelow.

In another aspect, the disclosure is directed to a system for isolating intact acellular particles the system comprising a station for separating, by using a size exclusion gradient, a biofluid sample containing intact acellular particles of different sizes into subpopulations of intact acellular particles, each respective subpopulation individually comprising a different size range of intact acellular particles, and a station for isolating a respective subpopulation. In one instance, the station for separating comprises a particle purification liquid chromatography column comprising size exclusion beads having different pore sizes, the size exclusion beads layered within the column to provide a gradient along the length of the column wherein the largest pore size is at the inlet of the column and the smallest pore size is at the outlet the column, the biofluid sample flowing through the column from the inlet to the outlet, as described herein; and the station for isolating can comprise a fraction collector. The system can further comprise a station for analyzing the respective subpopulation to determine size of the intact acellular particles therein, the concentration of the intact acellular particles therein, or both. The station for analyzing can comprise a UV-VIS spectrometer to obtain light scattering information, including absorbance by the respective subpopulation of light in the visible range of between about 400 nm to about 600 nm, as described herein. In another aspect, the disclosure is directed to an assembly for isolating intact acellular particles comprising, in combination, a particle purification liquid chromatography column having an inlet and an outlet and configured to flow therethrough a biofluid sample containing intact acellular particles of different sizes, the column comprising size exclusion beads having different pore sizes, the size exclusion beads layered within the column to provide a gradient along the length of the column wherein the largest pore size is at the inlet of the column and the smallest pore size is at the outlet the column; a fraction collector configured to receive an eluate from the outlet; and a UV-VIS spectrometer configured to obtain light scattering information on the elute, as described herein. The assembly can further comprising, in combination: an analyzer to determine the size of an intact acellular particle in the eluate, the concentration of an intact acellular particle in the eluate, the refractive index of intact acellular particles in the eluate, or all together.

The following Examples are illustrative of the disclosure and not limiting to same.

### EXAMPLES

### Example 1

This example describes a high-resolution chromatographic size-guided turbidimetry-enabled dye-free system for purification and analysis of intact acelluar particles comprising EVs and MC from biofluids, with semen as a model. The method employed herein is based on the principle of size exclusion using a column with gradient bead sizes. This gradient column, coupled with an automated fraction collector, permitted obtention of an unprecedented high-resolution separation of particles into fractions of various sub-populations. Furthermore, UV-Vis spectroscopy was employed to accurately identify the separated particles and calculate particle size and concentration using turbidimetry calculations. Validation of turbidimetry size measurements was made by TEM, and NTA measurements, while concentration was validated by fluorescence spectroscopy. Immuno blotting, RNA profiling and proteomics analysis provided compositional validation.

Materials and Methods:

Ethics: All experiments in this study were completed according to University regulations approved by The University of Iowa and Stony Brook University Institutional Review Boards (IRB). All participants were adults who provided written informed consent for semen samples, and all laboratory personnel were blinded to clinical data.

Biofluid samples: The University of Iowa and Stony Brook University Institutional Review Board (IRB) approved the use of human blood and semen specimens. All samples were received unlinked to any identifiers. All experiments were performed in accordance with the approved University guidelines and regulations. Whole, 2% fat, and fat free cow milk (Derle Hygrade) were purchased from Walmart. Conditioned media was collected from cells cultured in their respective media supplemented with 10% 18 h-ultracentrifuged EV-depleted FBS.

Samples processing: Seminal specimens from healthy men, collected by dry ejaculation were stored at −80° C. until used. The samples were thawed at room temperature (RT), differentially centrifuged at 500×g for 10 minutes, 2000×g for 10 min, and 10,000×g for 30 min to remove spermatozoa, leftover cells, and large materials, respectively. Samples were aliquoted either after pooling 3-6 samples or as individual donor aliquots and stored at −80° C. Blood samples from 4 healthy donors were collected in different anti-coagulant type tubes (K₂EDTA, Heparin, Citrate and no anti-coagulant). The samples were left undisturbed for 2 hours, and then centrifuged at 2,000×g for 10 minutes at RT. Serum and plasma were collected, centrifuged at 10,000×g for 30 minutes, and pooled by tube-type. 300 μl of each pool was used for separation. The rest of the samples were aliquoted and stored at −80° C. 20 ml of milk samples, with at least 10 days prior to expiration, were centrifuged in 50 ml falcon tubes at 10,000×g for 30 minutes, the fat layer was carefully removed and 1 ml was subjected to column separation. First void clean catch urine sample was collected from a healthy male, clarified by centrifugation at 2,000×g for 10 minutes, and 10,000×g for 30 minutes, before concentration 10 times from 40 ml to 4 ml using Amicon ultra centrifugal filter unit, 3000 Da, of which 1.5 ml was used for column separation. Cells, U1 (NIH AIDS reagent Program), 293T (ATCC), and MDA-MB-231 (ATCC) were cultured in 150×20 mm dishes for 3 days until confluency in complete media supplemented with 10% 18 h-ultracentrifuged EV-depleted FBS (Atlanta Biologics). 10 ml of each supernatant was clarified by differential centrifugation and concentrated (Pierce™ Protein Concentrator 3K MWCO, Thermofisher) to 1 ml and separated on the gradient column.

Size Exclusion Column (SEC) description and separation: An empty glass column of 100 cm length, 1 cm inner diameter, and 79 ml volume (Econo-Columns®, Bio-Rad, cat #7371091) was packed in-house at room temperature by gravity with a gradient of epichlorohydrin cross-linked dextran beads of various exclusion limit controlled by different degrees of cross-linking The beads are commercially available from Cytiva and sold under the trade name Sephadex (previously branded for GE Healthcare). The beads characteristics are described in Table 1. The beads were slowly packed from bottom to top after overnight swelling in ultrapure water, starting with G-10 (at bottom of column; outlet) and ending with G-100 (at top of column; inlet). 1× or 0.1× Phosphate Buffered Saline (PBS) was used as mobile phase. Fractions were collected in Greiner UV-Star® 96 well plates using a fraction collector (Gilson, FC204), with 6 drops per well. UV-Vis and fluorescence of the fractions were measured using a plate reader (Synergy H1, Biotek).

TABLE 1
Bead characteristics

| | | | Particle size | |
|---|---|---|---|---|
| | | | distribution range, | |
| | | % of | dry beads | |
| | Catalogue | column | (volume share | Exclusion |
| Bead type | number | length | within range %)* | limit (Da)^{&} |
| G-10 | 17-0010-01 | 5 | 40 to 120 (95%) | <7 × 10² |
| G-15 | 17-0020-01 | 7.5 | 40 to 120 (95%) | <1.5 × 10³ |
| G-25 fine | 17-0032-01 | 11 | 20 to 80 (97%) | 1 × 10³−5 × 10³ |
| G-50 medium | 17-0043-01 | 17.5 | 50 to 150 (98%) | >3 × 10⁴ |
| G-75 | 17-0050-01 | 24 | 40 to 120 (99%) | >7 × 10⁴ |
| G-100 | 17-0060-01 | 35 | 40 to 120 (98%) | >1.5 × 10⁵ |

*as determined in the certificate of analysis from the manufacturer
^{&}as advertised in the product specifications from the manufacturer at: https://www.cytivalifesciences.com/en/us/shop/chromatography/resins/size-exclusion.

Nano Tracking Analysis (NTA): Size distribution and particle concentration of purified fractions were determined using ZetaView (PMX 110, Particle Metrix). The system was calibrated using 100 nm Nanosphere™ size standards (3100A, Thermofisher). Samples were diluted to the appropriate concentration in filtered ultrapure water and measurements were acquired using ZetaView software v8.04.02. Shutter was kept at 70 and sensitivity was adjusted to 2-4 points below the noise level in an effort to capture the small particle. Measurements were taken in triplicates. For the zeta potential, samples were diluted in filtered PBS to the appropriate concentration for measurements as noted by the software (usually between 80,000 to 200,000 times) and measurements were taken in pentaplicate. Experiments were repeated at least three times with similar results.

Acetylcholinesterase (AChE) Assay: AChE enzymatic activity was measured as known in the art: briefly, 15 μl of each fraction were lysed in 0.5% Triton X-100 in a 96-well plate, to which was added a solution of 100 μl of a 1:1 volumetric ratio of 1.25 mM acetylthiocholine chloride (Sigma-Aldrich) and 0.1 mM 5,5′-Dithiobis2-nitrobenzoic acid (Sigma-Aldrich). 15 μl PBS was used as AChE negative control. Absorbance was read at 450 nm for 30 min at 37° C. every 5 minutes in a plate reader (Synergy H1, Biotek). Data are reported as the mean from triplicate wells and error bars are S.D. Experiments were repeated at least three times with similar results.

SDS-PAGE protein profiles: One microliter from each fraction of the preparation was withdrawn for SDS-PAGE separation, which was carried out on 4-20% Bis-Tris gel (Bio-Rad) for 120 min at 100 V. Gel was stained with Coomassie Blue. Because F3 and F4 contained low to no detectable levels of proteins, 20 μl of F3 and F4 were concentrated and loaded in separate lanes. Experiment was repeated at least three times with similar results.

Western blot: Primary antibodies against CD63, CD9 (mouse, Developmental Studies Hybridoma Bank, DSHB, Iowa City, Iowa, USA), CD81 (mouse, Proteintech, Rosemont, Ill., USA), TSG 101 (rabbit, Proteintech), HSP70 (rabbit, R&D systems, Minneapolis, Minn., USA), and Semenogelin-1 (SEMG-1, mouse, Santa Cruz Biotechnology, Dallas, Tex., USA) were used for western blot analysis. After incubation with primary and secondary (IRDye 800CW Donkey anti-Mouse/Rabbit IgG, LI-COR, Lincoln, Nebr., USA) antibodies, the membranes were imaged with LI-COR Odyssey Infrared Imaging System (LI-COR).

Transmission electron microscopy (TEM): TEM analysis of the isolated fractions were conducted as known in the art: briefly, carbon-coated copper grids were glow discharged to make the film hydrophilic (Pellco Easiglow, 0.2 mpar, 30 mA, 40s, negative), then ten microliters of F1-4 were applied to the grid and allowed to sit for 30 seconds. After removing the excess samples with filter paper, the grids were washed with distilled deionized water (ddH₂O) twice, followed by staining with 0.7% Uranyl Formate solution for 20 seconds. The grids were allowed to air dry before viewed. TEI Tecnail2 BioTwinG 2 electron microscope was employed to view the samples and an AMT XR-60 CCD Digital Camera system was used to capture the samples. Experiment was repeated three times. At least two images from each repeat were used in the particle size determination using ImageJ (NIH).

1-palmitoyl-2-oleoyl-sn-glycero-3-phosphocholine (POPC) and Oleic Acid (OA) vesicle preparation: The method is known in the art: briefly, 5 mM phospholipid solution was prepared by evaporating 150 μl of 25 mg/ml of POPC in chloroform (Avanti Polar Lipids, Alabaster, Ala.) under a stream of nitrogen in a glass vial. The POPC thin film was hydrated with 1 ml of 1×DPBS and tumbled overnight at RT on a rotary mixer. For OA vesicles, 32 μl of OA (Sigma Aldrich) were dissolved in 1 ml of NaOH (0.1 M) to form a 100 mM OA micelle solution, of which 50 μl were added dropwise to 1 ml of 1×DPBS to form a solution of 5 mM OA vesicles. Vesicles were tumbled overnight at RT on a rotary mixer. POPC and OA vesicles were extruded through various size polycarbonate membranes (50-1000 nm) using a mini-extruder (Avanti Polar Lipids) to form monodisperse unilamellar vesicles.

Naphthopyrene (NP) assay for total lipid concentration: Naphtho[2,3-a]pyrene (>98%) was purchased from TCI America and dissolved in DMSO at a stock concentration of 2.5 mM. Two μl of stock NP was added to 1 ml of clarified seminal plasma, for a final NP concentration of 5 μM, and the mixture was incubated on a rotary mixer at RT for 1 h before gradient column separation. In parallel, 5 mM phospholipid solution (POPC), was serially diluted, and NP was added for a final amount of 5 μM, corresponding to 0.1 mol % of POPC. Fluorescence (Ex/Em, 292/465) and absorbance at 280 nm, 400 nm, and 600 nm, and 650 nm were recorded for both the seminal plasma and the POPC standard curve. The standard curve data were fitted to a linear function for NP fluorescence and A₄₀₀ to infer the total lipid content in the seminal plasma fractions.

Vesicle size and concentration modeling: For core-shell structures such as vesicles, the scattering cross section depends on a set parameter whose equations are known in the art. For this example, the exact Lorenz-Mie solution was used as opposed to the Rayleigh-Gans-Debye approximation for two reasons: First, Lorenz-Mie solution applies to wider ranges of sizes, which fits the heterogenic nature of EVs, whereas Rayleigh scattering is only applicable in a narrow ranges of sizes where the particle radii should be significantly smaller than the wavelength of the scattered light. Second, the exact Lorenz-Mie solution is more favorable when studying charged particles, which is also the case of EVs that have been reported to be negatively charged. Thus, we applied the well=known Wang et al. model (Biophysical Journal 116:659-669) that was developed to calculate the scattering cross section of concentric vesicles with arbitrary size, lipid concentration, membrane thickness or number of layers. This model uses the open-source light-scattering package HoloPy (holopy.readthedocs.io/) and is available on GitHub with illustrative example (https://github.com/anna-wang/vesicle-turbidity). In this Example, the EV turbidity spectra for F1 wells was first calculated from the absorbance measured in the visible range (400-600 nm) with a 5 nm step using the following equation:

Calc.Turbidity₍₄₀₀₋₆₀₀₎=2.303×(Absorbance₍₄₀₀₋₆₀₀₎−BG)

whereas Absorbance₍₄₀₀₋₆₀₀₎ corresponds to spectra measured by the plate reader and BG is the background absorbance, mainly resulting from the plastic interference of the 96-well plate.

Next was inputted Calc.Turbidity₍₄₀₀₋₆₀₀₎ for each well of F1 together with its corresponding total lipid concentration that was calculated in the above section, and, for each input concentration was generated an array of Modeled Turbidity₍₄₀₀₋₆₀₀₎ spectra with a step of 5 nm for vesicles of size ranging from 40 to 300 nm, with a 1 nm step. This size range was chosen to encompass EVs of all sizes.

Subsequently computed was a cost function (CF₄₀₀₋₆₀₀) as follow:

$${CF}_{400 - 600} = {\sum }_{i = 0}^{n} \vert {y}_{i} - {\hat{y}}_{i} \vert$$

whereas n is the number of wells for which the data is input, i is the index of the well, yᵢ is the calculated turbidity based on the experiment, and ŷᵢ is the array of modeled turbidity for the same concentration as i. When CF₄₀₀₋₆₀₀ reached a minimum for a given i, the vesicle size for that i corresponded to that from the closest Modeled Turbidity₍₄₀₀₋₆₀₀₎).

Finally, with the hydrodynamic radius for each well now known, was calculated the vesicle concentration (N_{C}) using the following equation for hollow spheres:

$${N}_{C} = \frac{[ L ] {10}^{- 3} {N}_{A} {A}_{L}}{4 π [ {R}^{2} + {( R - {l}_{B} )}^{2} ]}$$

whereas L represents the lipid concentration (in M⁻¹), N_{A} is the Avogadro number, R is the radius of the vesicles, l_{E} represents the bilayer thickness (5 nm), l_{W} represents the thickness of the interlamellar aqueous phase (3 nm), and A_{L} denotes the area per lipid (0.627 nm²).

RNA bioanalyzer: 20 μl of each fraction were purified using RNeasy kit (Qiagen) with on-column DNase I digestion step. RNA was eluted with 14 μl of water and analyzed with Agilent 2100 Bioanalyzer on an RNA 6000 pico chip (Agilent Technologies, Santa Clara, Calif.) according to manufacturer's instruction. Experiment was repeated three times with three different biological replicates with similar results.

Nucleic acids denaturing PAGE: 500 μl of each fraction were used for nucleic acids extraction twice with phenol/chloroform/isoamyl alcohol (25:24:1), pH 8.0 (Thermofisher) and twice with chloroform. The aqueous phase was transferred to a new tube and the nucleic acids were precipitated with 300 mM sodium acetate pH 5.2 and 2.5 equivalent volume of Absolute Ethanol. After chilling for 1 hour at −86° C., precipitated nucleic acids were pelleted by centrifugation (19,000 g, 20 min, 4° C.) and the pellets were resuspended in 100 μl water. 18 μl of each nucleic acids solution was mixed with 2 μl of 10× DNase I reaction buffer (New England Biolabs), to which vehicle PBS, 2.5 units RNase A and 100 units RNase T1 (RNAse cocktail A+T1, Invitrogen), 1 unit DNase I (NEB), or RNase and DNase together were added and the tubes were incubated for 1 h at 37° C. After 1 hour. 20 μl stop solution (50% formamide 50 mM EDTA and 0.1% Bromophenol and 0.1% xylene cyanol) was added and samples were subjected to 8M urea PAGE. The gel was run at 1000V constant for 5 hours and then stained with Sybr Gold® stain (Thermofisher) for 20 minutes and visualized by UV at 254 nm.

Proteomic Analysis: Three seminal plasma pools (6 donors each) were clarified and 1.5 ml of each pool was purified on 100×1 cm gradient SEC column described above. Fractions 1-3 were concentrated under reduced pressure and quantified by the Bradford assay. 50 μg were denatured in 8M urea and 50 mM Tris-HCl, pH 8.0, reduced with 10 mM TCEP for 60 min at RT, alkylated with 2 mM iodoacetamide for 60 min at RT, and then diluted to 2M urea with 50 mM Tris-HCl, pH 8.0. Two micrograms of Trypsin Gold (Promega) was added for overnight digestion (18 h, 37° C.), and then the tryptic peptides were immediately desalted using Pierce C18 spin columns (Thermo Fischer Scientific) at RT. Peptides were eluted with 80% acetonitrile and 0.1% formic acid (FA), dried completely on a SpeedVac Concentrator and resuspended in 5 μl of 0.5% FA before loading onto a 3-phase MudPIT column (150 μm×2 cm C18 resin, 150 μm×4 cm strong cation exchange SCX resin, filter union, and 100 μm×12 cm C18 resin). The other LC-MS parameters are known in the art.

Peak Lists and Search Engine Parameters: Peak lists, protein identifications and database searches were conducted using BSI PEAKS Studio search engine software version 8.5 (Bioinformatics Solutions Inc., Waterloo, Ontario Canada). For label-free quantitation (LFQ), employed was the Q module of BSI PEAKS software which uses expectation—maximization algorithms on the eXtracted Ion Chromatograms (XIC) of the three most abundant unique peptides of a protein to calculate the Area Under the Curve (AUC) [82].

Sequence Databases: The Swiss-Prot UniProt Human non-redundant database (up000005640) which consisted of 20,303 annotated human proteins was used as the reference database (https://www.uniprot.org/uniprot). Enzyme specificity was fully tryptic with maximum 3 missed cleavages and maximum 1 non-specific cleavages. Modifications used were Carbamidomethylation (57.02) as fixed and Oxidized Methionine (15.99) as variable. Parent mass error tolerance was set as 20.0 ppm, and fragment mass tolerance set to 0.5 Da. Known contaminants to be excluded were identified and removed using the common Repository of Adventitious Proteins (cRAP) database version 1.0, release 2012.01.01 (https://www.thegpm.org/crap/). This is a listing of common laboratory proteins, including bovine serum albumin (BSA) and trypsin precursors, non-sample lab contaminants from dust and human sample handling and molecular weight standard proteins. Threshold score/expectation value: The BSI PEAKS peptide score (−10 lgP) was used for significance score of detection was for all peptide-spectrum search results. This is a derived score from the peptide-spectrum match (PSM) p-value. The protein level PEAKS score is the weighted sum of the −10 lgP PEAKS peptide scores. A PEAKS protein score of >=20 was used as the significance threshold for all database search results. For the label-free quantitation (LFQ), an additional threshold of XIC AUC of the 3 most abundant peptides of a protein to be >1e5. FDR was set to 0.1% at the peptide-spectrum match (PSM) level.

Data mining and visualization: KEGG pathways and GO terms were determined using WEB-based Gene SeT AnaLysis Toolkit 2019. Clustering Heatmaps were drawn using heatmapper. The clustering method used was the average linkage with Euclidean distance measurement applied to both rows and columns. Venn diagrams were obtained using Venny platform (v2.1).

Results:

Multi-bead gradient SEC column as prepared above were used to isolate EVs from a variety of samples: Size exclusion separation is based on the principle of size discrimination where large size molecules are excluded from the beads and flush-out directly, while small size molecules are included in the beads and hence travel a longer time through the column. Thus, the large size molecules elute in the void peak while the small size molecule elute in the latter peak (FIG. 1A). Optimization of the separation parameters such as the size and type of the beads, the length and width of the column, as well as sample injection volume can largely improve separation profile by resolving the inclusion and exclusion peaks, but it cannot generate any additional peaks. Only a gradient of bead sizes can allow generation of additional peaks, where a particular sized molecule can be included in one bead size and excluded from the subsequent beads (FIG. 1B). Seminal plasma was clarified from a pool of six donors by differential centrifugation (FIG. 1C) and separated equal aliquots on three Econo® columns (50 cm×0.5 cm) packed equally under atmospheric pressure with cross-linked dextran Sephadex™ beads G-10, G-100, or with a gradient of multi-beads (G-10, G-15, G-25, G-50, G-75 and G-100). Four distinct peaks were obtained from the multi-bead gradient separation, while G-10 or G-100 monosize-bead columns allowed only a two-peak profile (FIG. 1D). To validate the results and the capability and flexibility of the SEC column, the following experiments were conducted. First, different sized columns were tested; it was found that the resolution increased with the length of the column, as expected (FIG. 1E). Separation of seminal plasma from four individual donors was then tested it was found that the four-peak profile was donor-independent, but the ratios between the peaks were different (FIG. 1F). Blood plasma was separated from a pool of four donors (FIG. 1G) and it was found that, unlike the seminal plasma, blood plasma consistently resolved into a three-peak profile, with the majority of the components present in the first peak, a very small second peak, and a sharp third peak. This profile was independent of the type of the blood collection tube, although the no-additive blood serum profile showed a small fourth peak absent in the blood plasma profiles from the different anti-coagulant containing tubes. Different grades (whole, 2% fat and fat-free) of commercial pasteurized cow milk were then tested and it was found that milk, similar to seminal plasma, has a four-peak with the majority of the components present in the first peak (FIG. 1H). Urine was also profiled and showed a unique 3-peak profile. Compared to semen, blood, and milk that have different profiles from each other, human urine had also a unique 3-peak profile with very small first peak and large second and third peaks (FIG. 1I). The efficiency of the gradient column was validated by separating cell culture supernatants. To this end was used cultured U1 cells, a U937-derived pro-monocytic cell line that are chronically infected with HIV-1 (NIH AIDS reagent Program), 293T cells, which are embryonic kidney epithelial cells (ATCC), and MDA-MB-231 cells, which are metastatic breast cancer cells (ATCC) for three days in complete media supplemented with 10% exosome-free FBS. 10 ml of each supernatant were clarified and concentrated (FIG. 1C) and then separated on the gradient column. The cell culture supernatants came only with two peaks (FIG. 1J) and the absorbance profiles were different from those of the other biofluids. These results show that multi-bead gradient size exclusion separation can separate a variety of biological samples and that each biofluid tested had a unique characteristic absorbance profile. Since seminal plasma exhibits one of the most complex absorbance profiles, it was used as a prototype biofluid for characterization of the components in each of its four peaks, development of analytical algorithms, compositional, and functional studies.

With more specific reference to FIGS. 1A to 1J which relate to columns packed with gradient-size beads achieve higher resolution as compared to monosize bead columns. FIGS. 1A,1B show schematics describing that monosize bead separation (FIG. 1A) would end up with only two peaks, whereas separation with a gradient of bead sizes (FIG. 1B) may achieve a multi-peak resolution. In FIG. 1C is a schematic of the workflow for clarification crude EV mixtures from body fluids and cell culture supernatants by differential centrifugation before column separation. FIG. 1D shows seminal plasma from six healthy men that were pooled and clarified, and equal volumes (1 mL) were run on 50 cm×0.5 cm G-10, G-100, and gradient Sephadex® beads packed columns. Elution was carried with PBS, fractions were collected in 96-well plates and the UV-Vis absorbance measurements were recorded using a pleate reader. The profiles shown correspond to the 280 nm wavelength. FIG. 1E shows gradient separation profiles of seminal plasma on columns from different length. 1.5 ml aliquots from the same pool of clarified seminal plasma (Healthy, n=6) that were loaded on different length Econo® columns (20, 50, and 100 cm) packed with multi-size beads with the elution and collection carried under the same conditions. FIGS. 1F to 1J were samples purified on a 100×1 cm gradient column. Fractions were eluted with PBS, collected in 96-well plates and the UV-Vis absorbance measurements were recorded using a pleate reader. (FIG. 1F shows seminal plasma separation profiles (1.5 ml) from 4 individual healthy donors. FIG. 1G shows blood plasma and serum separation profiles. Blood was collected from 4 donors in different collection tubes and clarified plasmas and serum were pooled by tube type. 300 μL of each pool were used for separation. FIG. 1H show commercial cow milk separation profiles. Whole, 2% fat, and fat-free cow milk was purchased from Walmart, clarified by centrifugation at 10,000 g for 30 minutes and 1 mL of each clarified sample was used. In FIG. 1I, 40 ml of first void clean catch urine sample was collected from a healthy male, clarified, concentrated 10 times to 4 ml (Amicon™ ultra centrifugal filter unit, 3000 Da), of which 1.5 ml was used for purification. FIG. 1J shows the following: U1, 293T, and MDA-231 cells were cultured in 150×20 mm dishes in media supplemented with 10% exosome-depleted FBS for 3 days and 10 ml of supernatants were clarified by differential centrifugation and concentrated (Amicon™ ultra centrifugal filter unit, 3000 Da) to 1 ml before purification.

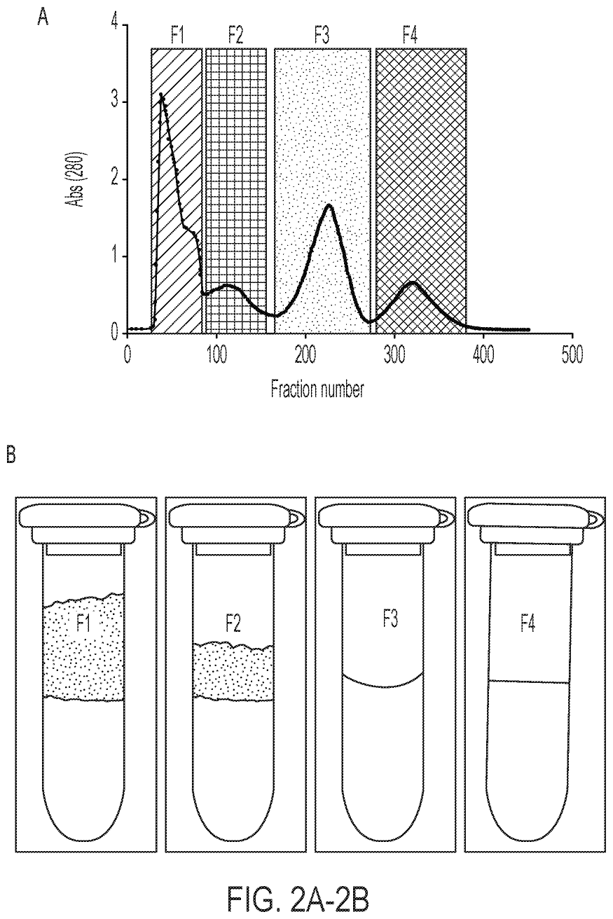

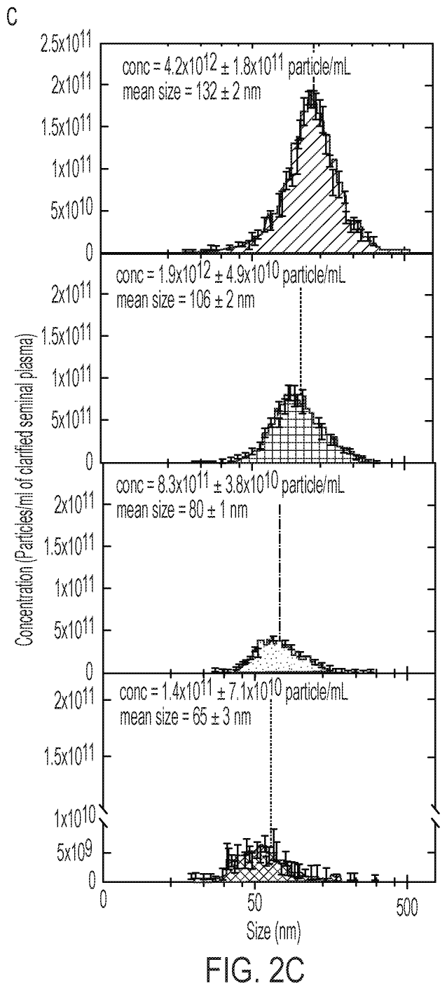

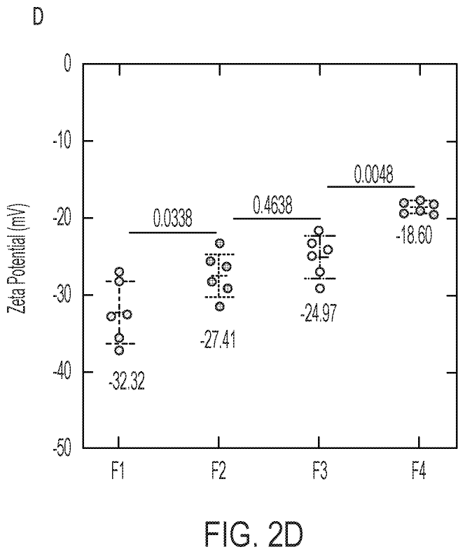

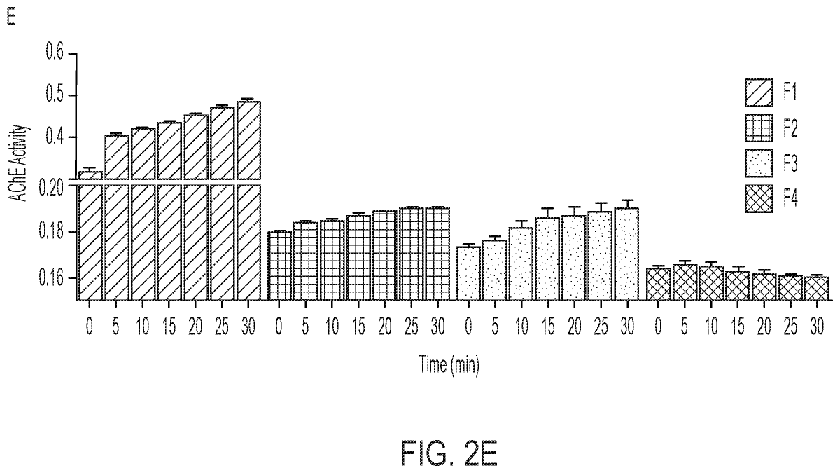

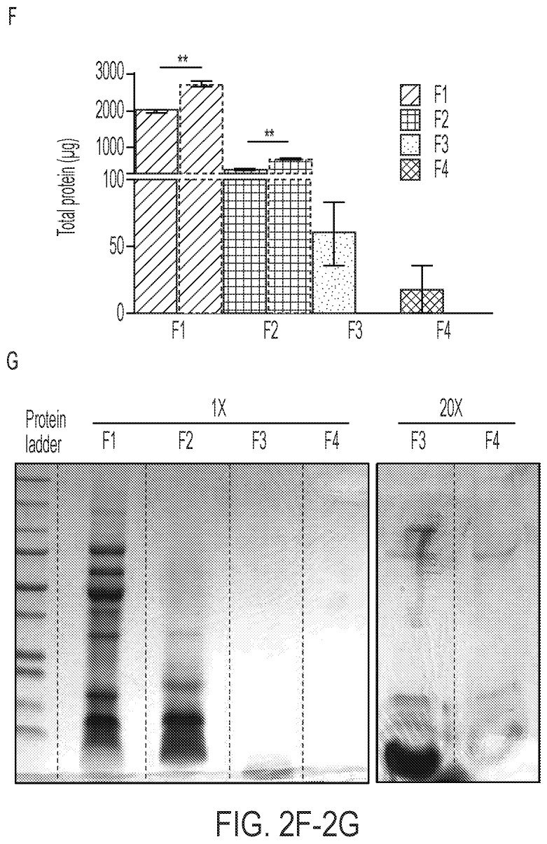

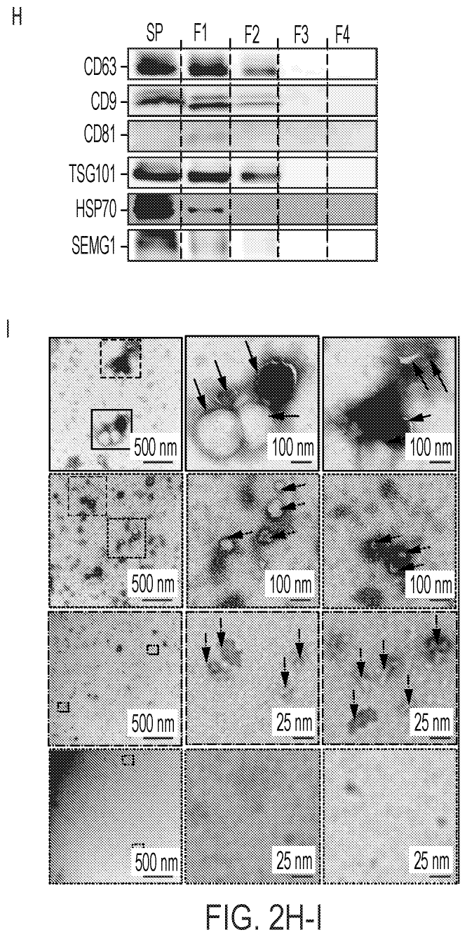

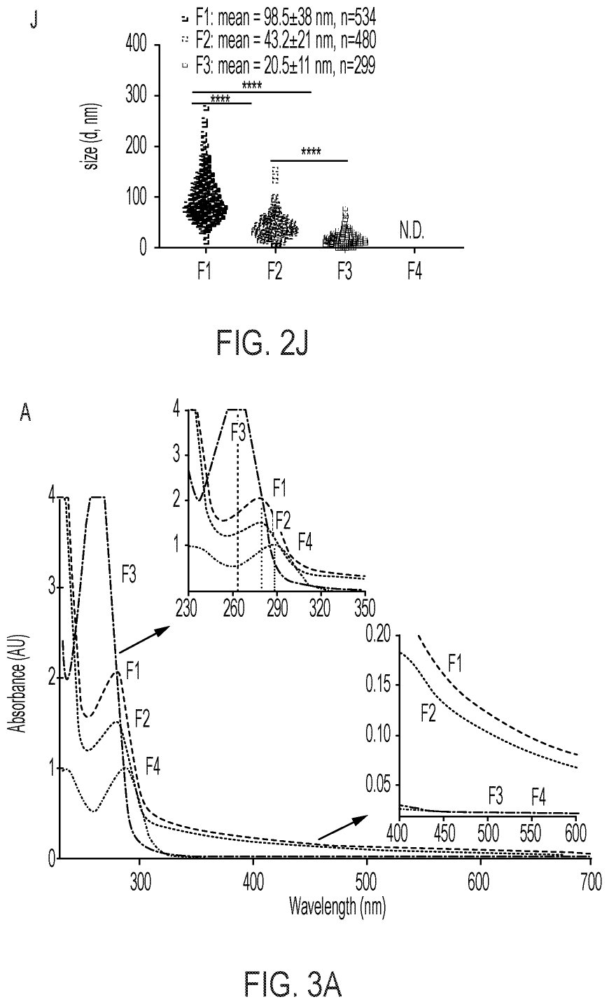

Multi-bead gradient SEC column isolates different EV-subpopulations and MCs from seminal plasma: Post-column fractions were collected in 96-well plates and were binned into four pool fractions (F) named F1-F4, frozen and concentrated under reduced pressure (FIG. 2A). The volume of the fractions was then adjusted to the input volume; hence the components' concentration in each fraction was not different from the original seminal plasma concentration (FIG. 2B). Qualitative clues about the components of each fraction can be inferred visually where F1 and F2 had foamy top layers (more pronounced in F1), an indication of the presence of lipid vesicles; F3 was yellowish, which indicated potential presence of leftover urobilin from urine, since semen and urine both travel through the urethra, and F4 was clear which indicated the presence of colorless components, such as fructose and minerals. F3 always contained visible particulates that never dissolved after readjustment to the input volume. Nano-Tracking analysis (NTA) of F1-F4 validated the size-guided separation where the mean size of the vesicles decreased gradually (FIG. 2C, X axis). The concentration of the vesicles decreased as well where F1-F4 contained ˜59%, ˜27%, ˜11% and ˜2% of the vesicles, respectively (FIG. 2C, Y axis). The zeta potential was also different between the fractions, with a net negative surface charge decreasing from F1 to F4 (FIG. 2D). Acetylcholine esterase (AChE) activity assay of the fractions showed that F1 contained most of the AChE activity, F2 and F3 contained residual activity and F4 had no detectable activity (FIG. 2E). This differential enrichment of AChE in the pooled fractions is consistent with findings that AChE test, although convenient, cannot be used for EV detection since acetylcholine may not be exclusive to EVs. The protein levels in the fractions were assayed in the presence and absence of triton. The results in FIG. 2F showed that majority of the proteins (˜80%) were in F1, and ˜17%, ˜2% and ˜1% of the proteins were distributed in F2-F4, respectively. Addition of triton significantly increased the levels of proteins in F1 and F2 but not in F3 or F4, confirming the presence of vesicles in F1 and F2, which had opened upon triton treatment and released their protein cargo, whereas F3 and F4 may not contain vesicle-encased proteins. F1-F4 were then separated on a SDS-PAGE which showed that indeed F1 and F2 contained most of the proteins, while F3, despite its high absorbance reading at 280 nm (FIG. 2A), only contained small (˜10 KDa) peptides and F4 contained no proteins, even after loading 20 times more sample (FIG. 2G). Western blot analysis of known EV markers (CD9, CD81, CD63, HSP70 and TSG101) also showed enrichment of EVs in F1 and F2 and their absence in F3 and F4 (FIG. 2H). Finally, negative stain TEM imaging (FIG. 2I) was employed to further confirm the identity of the fractions. Structural F1 and F2 contained large and small membranous vesicles respectively. Unlike these membranous vesicles, F3 was enriched in membraneless structures that are ˜20 nm in size with defined sharp edges, while F4 contained neither vesicles nor any detectable feature. Quantitative analysis of the TEM images confirmed enrichment of particles in F1, F2, and F3, in that order, with none in F4 (FIG. 2J). Given their size, presence or absence of membrane, we named F1 large SEV (SEV_{L}), F2 small SEV (SEV_{S}), and F3 membraneless condensates (MCs). These results confirmed the presence and successful isolation of SEV_{L} and SEV_{S}, as well as novel MCs from seminal plasma. The archetypal features of seminal vesicles maybe connected to their function.

With more specific reference to FIGS. 2A to 2J which relate to human seminal plasma and distinct EV subpopulations contained therein. FIG. 2A shows collected fractions in 96-well plates were pooled into four fractions, frozen and concentrated under reduced pressure, and volume was adjusted to the input volume. To control for the amount of salts added to the samples, purification (which dilute the samples 10 times) was performed using 0.1×PBS buffer and the volume of the concentrated samples was re-adjusted with ultra-pure water. Thus, the seminal plasma was separated into four fractions in 1×PBS buffer, without enriching or diluting the inherent components of each fraction. FIG. 2B provides representative pictures of the four fractions after volume readjustment. FIG. 2C shows size and concentration of F1 to F4 by NTA. Error bas are SD of triplicate measurements. FIG. 2D shows zeta-potential as measured by NTA. Error bas are SD of pentaplicate measurements. Ordinary one-way ANOVA test (Tukey's test) was used to determine the differences between F1-F4. Exact p-Values are given in the figure. FIG. 2E shows AChE enzymatic activity. Error bas are SD of triplicate wells. FIG. 2F shows total protein in fractions F1 to F4 as quantified by Bradford assay in the presence and absence of triton. Error bas are SD of duplicate measurements. Unpaired t-test with Welch's correction was used to determine the differences between the groups. **, p<0.01. FIG. 2G are representative of SDS-PAGE showing the protein profile of fractions F1 to F4. FIG. 2H shows Western blot of exosome markers. Loading was done by equal volume. FIG. 2I are representative negative-stain TEM images of fractions F1 to F4. The middle and right columns correspond to zoomed areas in the left column indicated by the open squares. Scale bars=500 nm for left, and 10 nm for close-up images. Experiment was repeated at least three times with similar results. FIG. 2J shows TEM-based mean particle size determined with Image J. At least three representative images from each of the three experiments was used for quantification. Ordinary one-way ANOVA test (Tukey's test) was used to determine the differences between F1-F4. *, p<0.0001. ND, not determined.

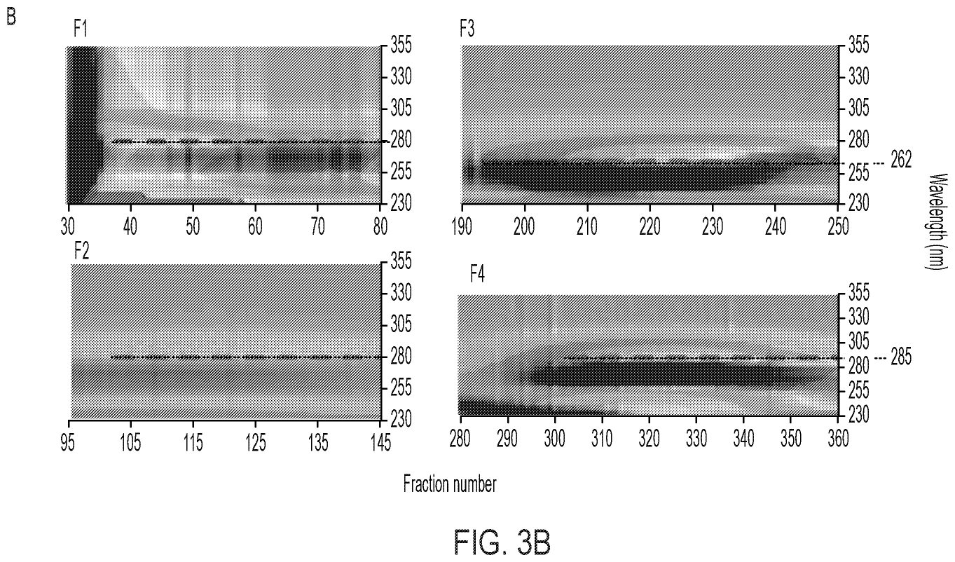

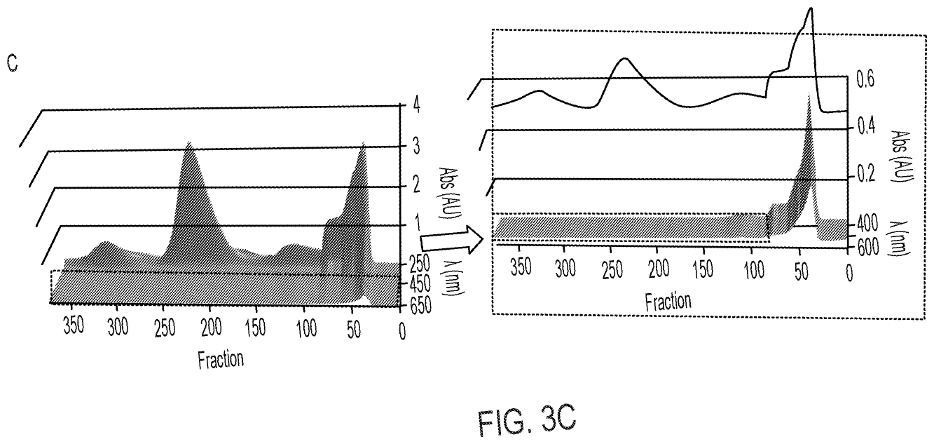

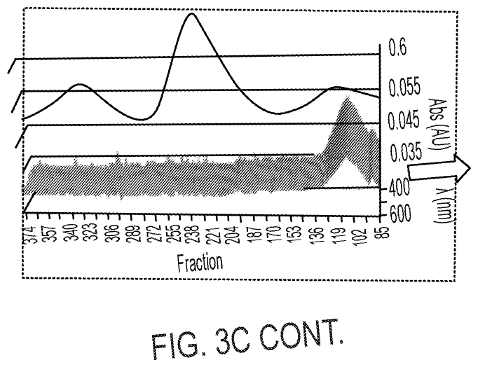

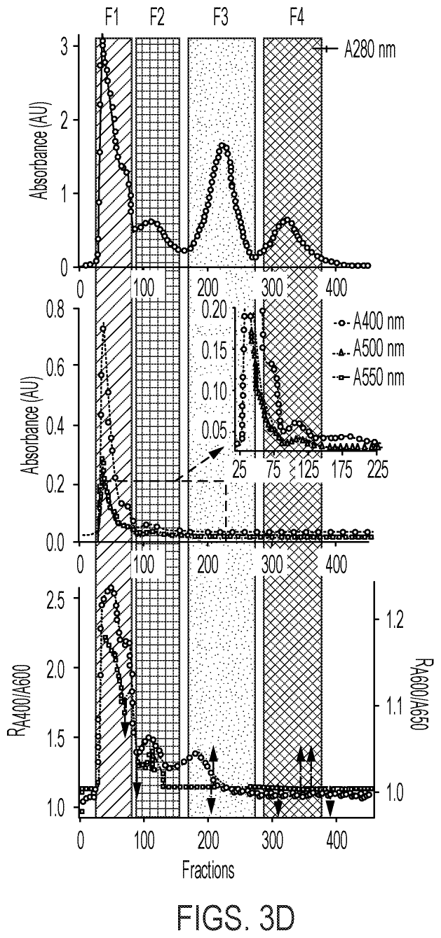

UV-Vis analysis identifies the molecular components of the purified seminal fractions: Absorbance at 280 nm (A₂₈₀) has been used to determine the presence of EV during size exclusion chromatography; however, this wavelength is not ideal for EV detection since free proteins may be confounded for EVs. Monitoring the EV separation in the turbidity range (400-600 nm) was chosen instead for EV detection. The hydrophobic interlayer of the EV membrane scatters light in the visible spectrum range of light making the lipid vesicle-containing solution turbid. The UV spectrum range (190-350 nm) is also essential as it contains critical information regarding the nature, the concentration, and the purity of organic molecules. Thus, the full UV-Vis spectrum of fractions F1-F4 was measured (FIG. 3A). The shoulder in the turbidity range of F1 and F2 (FIG. 3A) was indicative of the presence of membranous vesicles, while F3 and F4 were determined to be membraneless given the absence of the turbidity range shoulder in the spectra (FIG. 3A, bottom inset). In the UV range, F1 and F2 peaked at 280 nm, indicating protein decoration of the particle surfaces. F3 blue-shifted to ˜262 nm which pointed to a potential presence of free nucleic acid whereas F4, which contained the smallest molecules, red-shifted to ˜285 nm which pointed to the presence of small peptides and minerals known to be present in seminal plasma. This UV-Vis spectral analysis corroborated the results in FIG. 2 and validated that F1 and F2 were membrane-containing EVs fractions, and also provided new information in which MCs in F3 fraction may be enriched in free-nucleic acid aggregates.

Three dimensional (3D) UV-Vis profile validates components of the purified seminal fractions: In order to test and extend the ranges of the system in depicting the nature of the biofluid-derived components, 3D UV-Vis measurements were employed (fraction/wavelength/intensity) in the UV-range (FIG. 3B) and visible range (FIG. 3C). From this analysis, vesicles-containing wells were identified, which spanned over wells 29-125 (FIG. 3C, red arrow and enlargement). Furthermore, and particularly in the visible range, the following ratios, R₁=A₄₀₀/A₆₀₀ and R₂=A₆₀₀/A₆₅₀, were defined as a qualitative turbidity index. The ratios are used concurrently to rule-in or rule-out the presence of vesicles (FIG. 3D). The indices must superimpose for a given well to contain vesicles. In contrast, if one index (often R₁) shows a peak that is absent in the other, it is an indication of the presence of impurities, such as colored materials. Indeed, in the example presented in FIG. 3D, F1 and F2, which contain EVs, exhibit similar R₁ and R₂ profiles (FIG. 3D bottom), whereas F3, which lacks EVs, exhibited a peak only in R₁, indicating the presence of a non-vesicular turbidity-exhibiting material (FIG. 3D bottom). Based on this definition, pinpoint vesicle-containing wells with high accuracy and without the need for full spectra measurements. As shown, 3D UV-Vis profiling during separation can be used to identify a wide range of biomolecules in a sensitive and non-invasive manner.

With more specific reference to FIGS. 3A to 3D which relate to UV-Vis spectroscopy for characterizing EV subpopulations. FIG. 3A shows UV-Vis spectra of fractions F1-F4. Top and bottom insets represent enlarged graph of UV and visible spectra, respectively. Scans were performed from 230-700 nm with 2 nm intervals. FIG. 3B shows a contour view of 3D UV spectra of fractions F1-F4 prior to pooling showing a 280 nm peak for F1 and F2, 262 nm peak for F3, and 285 nm peak for F4. FIG. 3C shows a 3D-surface plot of fractions F1-F4 spectra in the turbidity range showing the presence of a shoulder in F1 and F2 that is absent in F3 and F4, despite the high peaks in the UV range. The inset line corresponds to the 280 nm profile. Middle and right represent zoomed areas in the plot, indicated by a dashed rectangle. B and C plots were drawn in Microsoft Excel 2019. FIG. 3D shows A₂₈₀ profile (top) as compared to A₄₀₀, A₆₀₀ and A₆₅₀ profiles (middle). Bottom graph depicts R₁ and R₂ ratios (R₁=A₄₀₀/A₆₀₀, left axis and R₂=A₆₀₀/A₆₅₀, right axis).

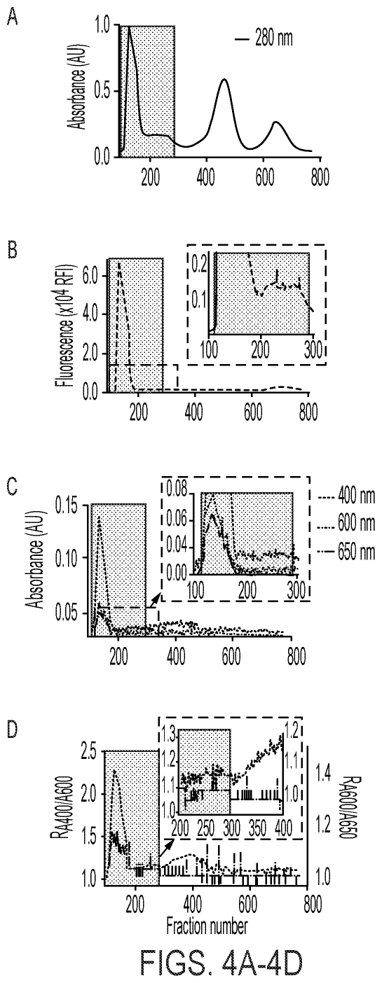

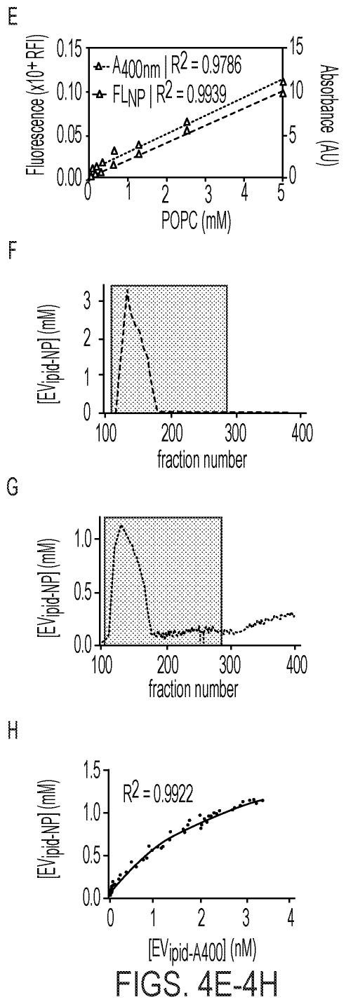

UV-Vis analysis accurately determined the lipid concentration of purified EVs: Turbidity is converted into a quantitative parameter of EV particle number. To this end was added 1 μM of Naphtho[2,3-α]pyrene (NP), a polycyclic aromatic hydrocarbon that fluoresces only when embedded in the lipid bilayer to clarified seminal plasma. After brief tumbling at room temperature using a rotary mixer, the seminal plasma was purified and both absorbance and fluorescence (FIG. 4A-C) were monitored during the purification procedure. While raw turbidity profiles (A₄₀₀, A₆₀₀, A₆₅₀) will not rule-in the presence of vesicles beyond fraction number ˜190 (FIG. 4C), NP fluorescence profile indicated that vesicle-containing wells extended until fraction number ˜290 (FIG. 4C inset). On the other hand, R₁ and R₂ analysis revealed that fractions up to ˜290 exhibited a peak slightly above the background noise, albeit small (FIGS. 4D and 4D inset). Beyond fraction number 290 (300-400), the increase of R₁ without an increase in R₂ indicated the presence of non-vesicular material, an interpretation corroborating the lack of fluorescence in these fractions (300-400) (FIG. 4D inset). The difference in the low detection range between turbidity and fluorescence arises because background in fluorescence is generally less pronounced compared to absorbance and because fluorescence is often more sensitive than absorbance with a higher dynamic range. Nevertheless, it is possible to identify vesicle-containing wells using turbidity calculations. Subsequently, to attribute both turbidity and NP measurements to an absolute lipid concentration, a solution of known concentration of synthetic 1-palmitoyl-2-oleoyl-sn-glycero-3-phosphocholine (POPC) vesicles, to which NP was added at a concentration of 1 μM, is used and the mixture is serially diluted to generate a standard curve (FIG. 4E). Both A_{400-POPC} and NP_{POPC} strongly correlated to a linear regression (R²=0.9786 and 0.9939, respectively). Total lipid concentration per fraction was thus inferred from the corresponding linear function for fluorescence (FIG. 4F) and turbidity (FIG. 4G). Although there was an underestimation in the concentrations determined by turbidity method as compared to the NP fluorescence, the regression between the data was strong with a R² of 0.9922 (FIG. 4H). Finally, the data best correlated to an exponential fit that highlights the difference in the dynamic ranges of detection between the two methods. Taken together, the data presented here provide an accurate way to determine EV lipid concentration using NP fluorescence, but also, monitoring turbidity during EV isolation could allow accurate and reproducible dye-free EV lipid quantification. Note that the turbidity and NP analyses were not applied to MCs because these fractions are membraneless.

With more specific reference to FIGS. 4A to 4H which relate to NP Fluorescence and turbidity measurements to quantify total EV lipid concentration. 1 ml of clarified seminal plasma was incubated with naphthopyrene dye at 1 μM final concentration and, after brief tumbling at room temperature, the sample was purified on a gradient SEC column and fractions were collected in 96 well plates (4 drops/well). FIGS. 4A-4C show separation profiles at (FIG. 4A) A₂₈₀, (FIG. 4B) NP fluorescence, and (FIG. 4C) A₄₀₀, A₆₀₀, A₆₅₀. Inset in FIGS. 4B and 4C represent a zoomed area, indicated by a dashed rectangle. The gray bar represents the data used in the subsequent analysis. FIG. 4D shows R₁ and R₂ ratio profiles with good agreement over the EV range (100-290) and disagreement beyond fraction number 300. The inset displays the zoomed area indicated by a dashed rectangle. In FIG. 4E, 5 mM POPC vesicles were prepared by the thin film rehydration method in PBS, to which 1 μM naphthopyrene dye was added. A standard curve was prepared and both A₄₀₀ and NP fluorescence were recoded. Data were fit to a simple linear regression using Prism software. FIGS. 4F-4G show calculation of the total lipid concentration using the corresponding equations of the linear fit calculated in FIG. 4E. FIG. 4H shows non-linear regression between the total lipid concentrations as determined by turbidity and NP fluorescence showing good agreement between the two methods.

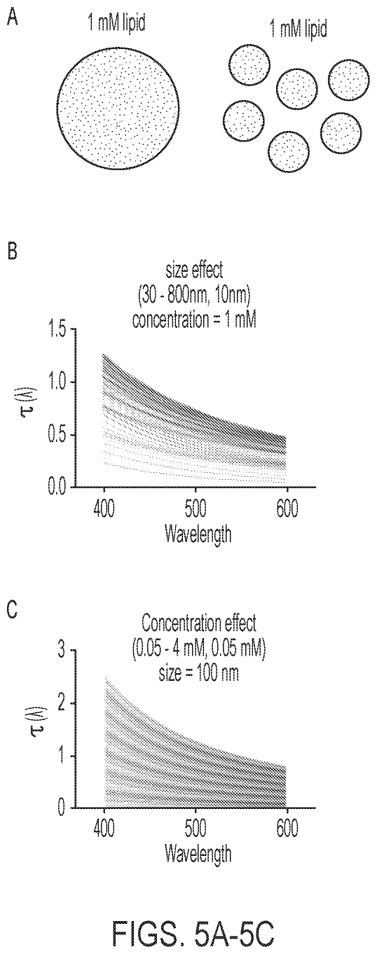

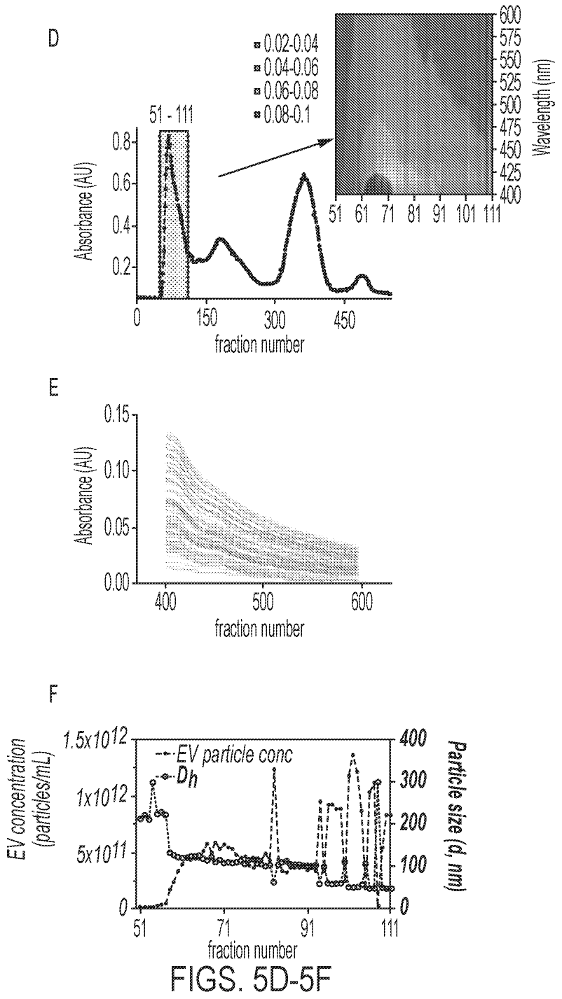

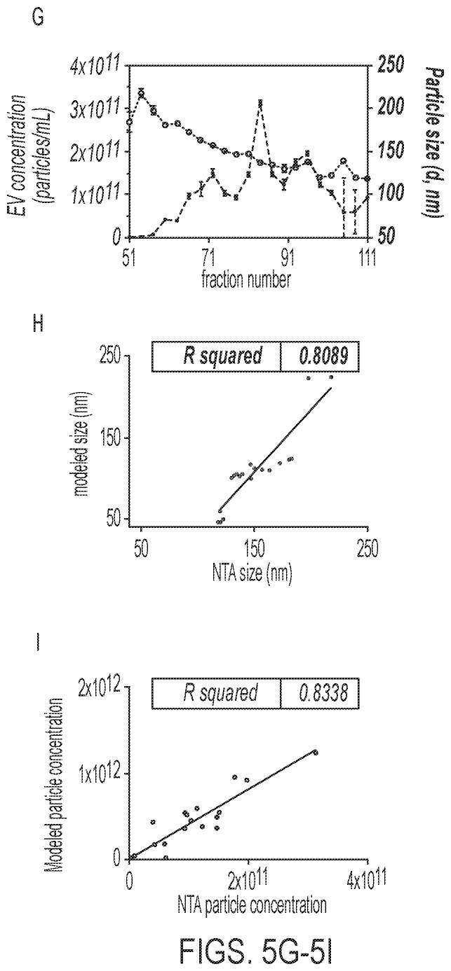

UV-Vis analysis accurately determines the size and particle number of purified EVs: As demonstrated above, the application of turbidimetry is an effective, dye-free way to accurately determine lipid concentration in fractions during EV purification. But total lipid concentration alone does not permit particle number calculation without information about the particle size (FIG. 5A). The UV-Vis measurements of the disclosed method can be coupled with a high-resolution size-guided separation to yield populations of monodisperse particles per fraction. This monodispersity implies that the particle size is directly inferable from the turbidity spectra whether by applying the exact Lorenz-Mie solution or the Rayleigh-Gans-Debye approximation, without the need to invoke a distribution function, such as the log-normal Gaussian distribution. Turbidity represents the attenuation of incident light due to light scattering and determination of particle size spectrophotometrically. Turbidity measurements for size determination has been previously applied to nanoparticles, liposomes, and other lipid vesicles such as protocells, but not to EVs. Here, the Lorenz-Mie turbidity model as discussed above was applied to calculate the hypothetical turbidity spectra for lipid vesicles of different concentrations and different sizes (FIGS. 5B-5C), thus creating a matrix library of ˜10,000 hypothetical spectra. An assumption in the library calculations was an EV membrane thickness of 5 nm, and an EV lamellarity of 1 for biological membranes, and used for medium and EV refractive indices the equations derived for water and egg PC, respectively, as known in the art. Then calculated was the turbidity spectra of F1 encompassing wells 51-111 by removing the background and multiplying the measured absorbance values by 2.303 (FIG. 5E). Using the calculations from the above section, the determined the total lipid concentration per well was determined and which was used to determine the EV particle size per fraction as well as the EV particle number (FIG. 5F). To validate these calculations, NTA measurements was conducted on the individual fractions (FIG. 5G), which also showed decreasing size as expected from a size exclusion chromatography. As for the particle concentration, NTA and turbidity calculations yielded similar overall numbers. The linear regression between the two methods for particle size and concentration determination showed good correlation with R squared of 0.8089 and 0.8335, respectively. Comparative analysis of NTA versus turbidity size data showed that the NTA sizes ranged from 217 to 118 nm, whereas the turbidity sizes spun over wider range from 224 to 47 nm (FIGS. 5E, 5G). As for particle concentration, turbidity calculation counted 3.06×10¹³ particles ˜4.7 fold larger as compared to NTA volume-adjusted total particle number which was 6.58×10², indicating that the turbidity model may be more sensitive and suitable for measuring EV particle number.

With more specific reference to FIGS. 5A to 5I, which relate to turbidity measurements for determining EV particle size and concentration. FIG. 5A is a visual depiction showing that same total lipid concentration in a solution can form endless combinations of heterogeneous size and particle concentration. FIGS. 5B-5C show representative modeled spectra for hypothetical hollow spheres with varying (FIG. 5B) lipid concentration at a fixed particle size of 100 nm, or (FIG. 5C) particle size at a fixed lipid concentration of 1 mM. FIG. 5D shows separation profiles showing (A) 280 nm absorbance. The Inset represents the 3D contour view of F1 area, indicated by a gray lane (wells 51-111), and which was used in the subsequent calculations. FIG. 5E shows measured turbidity spectra of F1 area after removal of background and transformation of plate reader measured absorbance into turbidity. FIG. 5F shows EV particle concentration and hydrodynamic diameter (Dₕ) as calculated from the measured turbidity spectra. FIG. 5G shows particle size and concentration of individual fractions determined by NTA as an independent method of validation. (H-I) Linear regression for (FIG. 5H) size and (FIG. 5I) particle concentration between the turbidity model and NTA data showing good agreement between the two methods.

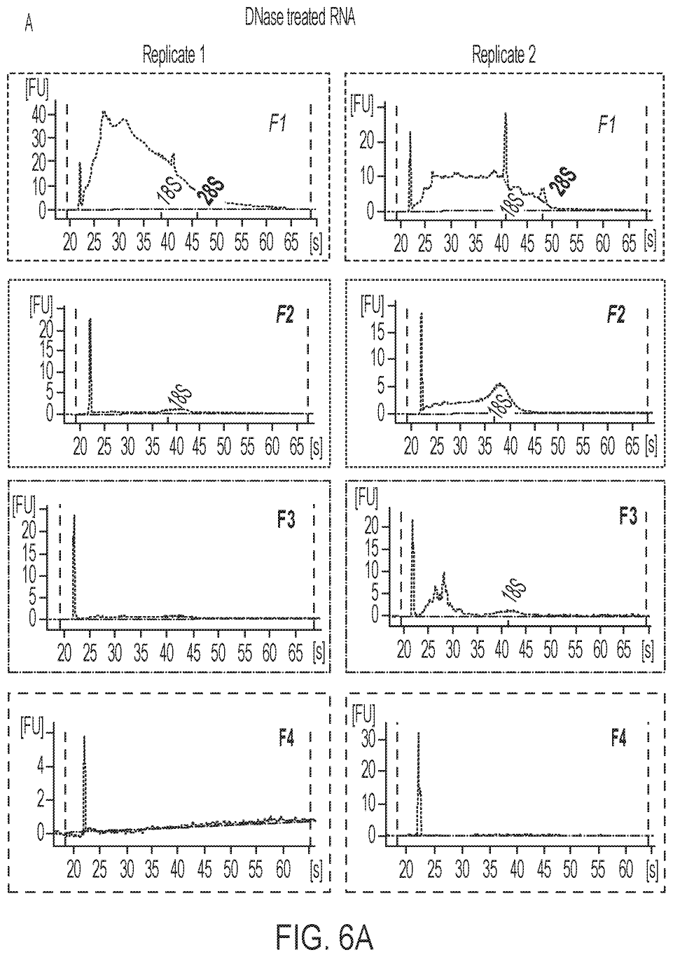

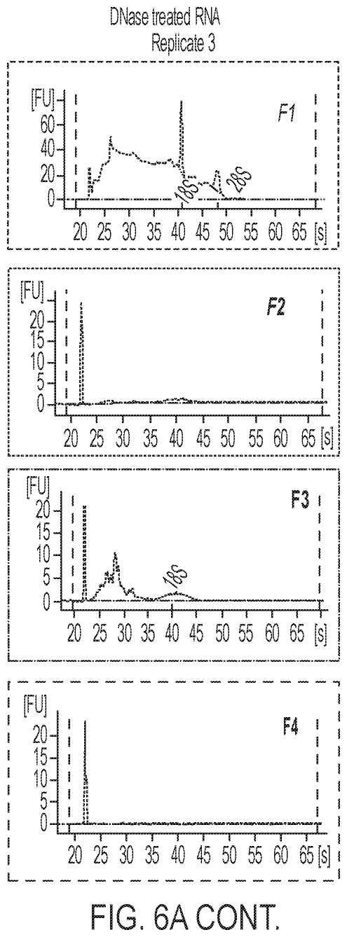

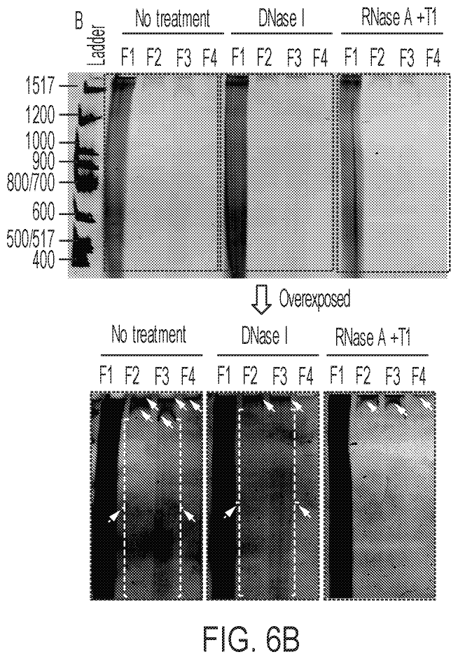

High-resolution chromatographic size-guided turbidimetry-enabled dye-free system permits identification of EV- and MC-associated cell-free nucleic acids (cf-NA): Human semen derived EVs contain a repertoire of small non-coding RNA and seminal RNA plays critical roles not only in sperm maturation and fertilization, but also in embryo preimplantation and early embryogenesis. Human seminal EVs were also demonstrated to contain DNA fragments ranging from ˜500 to ˜16,000 bp, but DNA (or RNA) species fractionation was not yet achieved. The method of the disclosure was used to separate different nucleic acid species. RNA was then extracted from F1-F4 using RNeasy® with the optional on-column DNase I digestion performed, and eluted RNA samples subjected to Agilent Bioanalyzer RNA profiling. The RNA profiles (FIG. 6A) show decreasing size of RNA species with the separation, where large RNAs including 18S and 28S rRNAs are enriched in F1, medium-size RNA enriched in F2, while small RNA (typical miRNA sized species) are enriched in F3, and F4 was RNA-free. In a separate experiment, cfNA in F1-F4 were isolated by phenol/chloroform/isoamyl alcohol (25:24:1) at pH 8 and ethanol precipitated. Purified cfNA were subjected to a 8% denaturing PAGE. The results show that F1-F4 are differentially enriched in cfNA, with F1 containing most of the cfNA, followed by F3, F2 and then F4 (FIG. 6B). Furthermore, specific bands (indicated by red arrows, overexposed panel) disappeared upon RNase-free DNase treatment and appeared in the RNase-treated lanes demonstrating the cfDNA content of F2-F4. In contrast, blue-arrows indicate bands that persisted after DNase treatment and disappeared upon RNase treatment, pointing to the presence of RNA species in F2 and F3 as well. Finally, for F1, which contains over 90% cfNA, it is clear that it carries both RNA and DNA species. It was not possible to determine the enrichment of a species over the other, however the smear in F1 decreased more in the RNase than in the DNase treatment (FIG. B, normal exposure) suggesting that F1 carries more RNA than DNA cargo. It is important to note, that this gel does not show cfNA species smaller than 400 bp in length. Nonetheless, the bioanalyzer profiles (FIG. 6A) demonstrate that method of the disclosure provides separation of biofluids into fractions containing EV sub-population or MCs prior to total RNA or DNA purification. This combination allows fractionation of RNA species, which no other RNA isolation kit has achieved thus far.

With more specific reference to FIGS. 6A and 6B which show that cell-free nucleic acids (cf-NA) are differentially enriched in EVs and EV-free seminal plasma. FIG. 6A shows RNA Bioanalyzer profiles of DNase I-treated RNA isolated from F1-F4. Experiments were repeated three times with different biological samples. FIG. 6B shows denaturing PAGE of cf-NA isolated by phenol/chloroform extraction from F1-F4, untreated or treated with DNase I, or with RNase cocktail (RNase A+RNase T1). Gel was run at 1000V for 5 h before incubation in Sybr-Gold solution in the dark for 10 minutes and visualized under UV. Gel was imaged in normal exposure and overexposure settings. Red and blue arrows denote DNA and RNA bands, respectively.

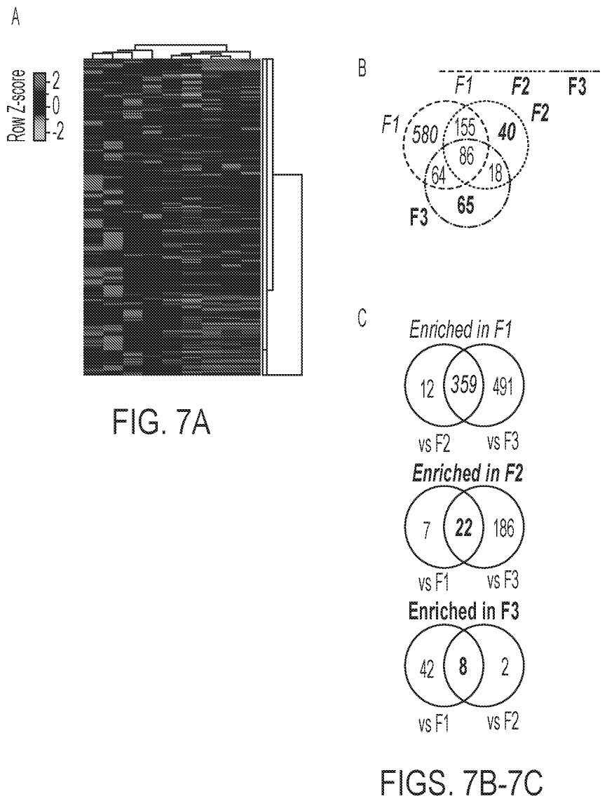

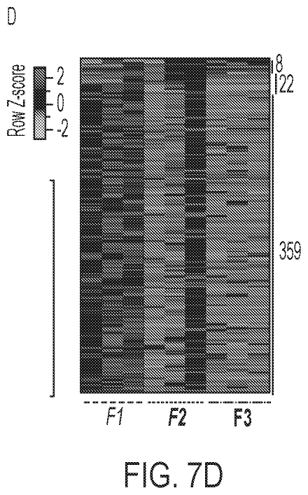

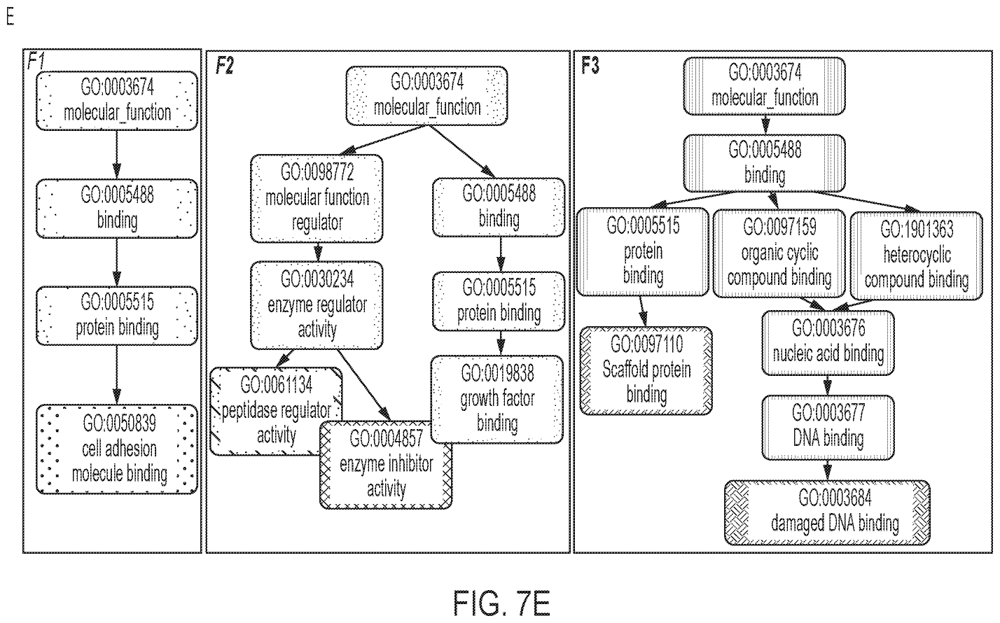

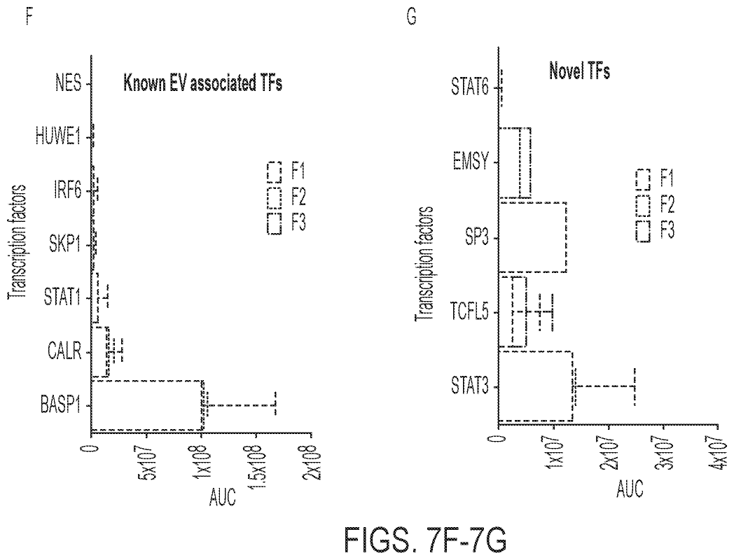

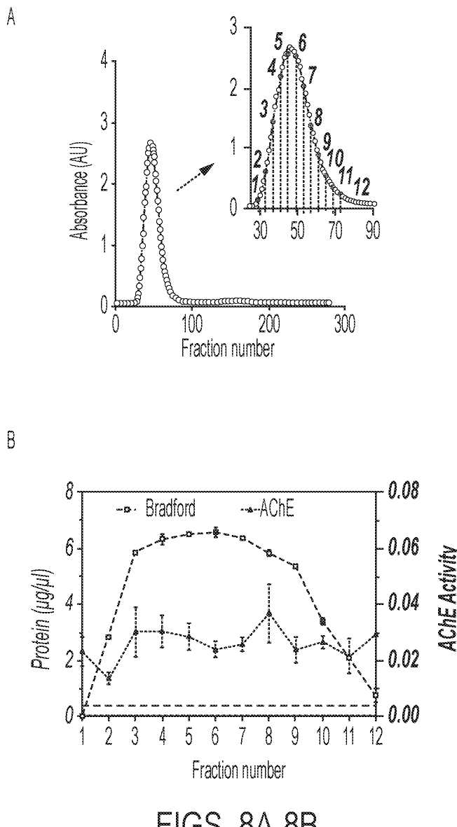

High-resolution chromatographic size-guided turbidimetry-enabled dye-free system permits identification of EV- and MC-associated proteins: Proteomics analysis can be used to identify seminal proteins that are enriched in SEVs and those that are mostly present in EV-free seminal plasma. Conducted was MudPIT analysis of F1-F3 from three biologically independent pools of seminal plasma. F4 analysis was not performed, since in the separation profile and characterization of seminal plasma F4 consistently contained no detectable proteins. The spectral count (SpC) data identified a total of 2178 proteins with at least one unique peptide (FIG. 7A), of which 1204, 516 and 466 were common to all three biological replicates of F1, F2 and F3, respectively. Of those proteins, common and exclusive proteins were distributed among the 3 fractions (FIG. 7B). To identify the enrichment pattern of the seminal plasma proteins in the fractions, the cutoff at the protein level was made to at least 2 unique peptides and Ordinary Two-way ANOVA tests were performed between the different fractions and controlled for the false discovery rate (FDR) which was set to 0.05, using the original method of Benjamini and Hochberg. By this criteria, 359, 22 and 8 proteins were differentially present in F1, F2 and F3 respectively (FIGS. 7C, 7D). No-redundant molecular function GO analysis of these differentially present proteins revealed that F1 is enriched in proteins involved in cell adhesion molecule binding, F2 enriched in processes involving growth factor binding, enzyme inhibitor activity, and peptidase regulator activity, whereas F3 is enriched in proteins involved in scaffold protein binding and damaged DNA binding (FIG. 7E). Furthermore, previously identified EV-associated transcription factors (TFs) as well as five novel cell-free TFs (namely, STAT3, STAT6, TCFL5, EMSY and SP3) were uncovered within EVs and MCs (FIG. 7F-G). This finding suggests that more regulatory proteins could be present with potential precise function in the intended recipient cells and that further dissection of the cell-free seminal plasma fraction may facilitate their identification.

With more specific reference to FIGS. 7A to 7G which show that extracellular proteins are differentially enriched in EVs and EV-free seminal plasma. FIG. 7A is a cluster heatmap of the 2178 seminal plasma proteins identified above. FIG. 7B is a Venn diagram of the common and distinct proteins in F1-F3 as determined by the spectral count (SpC) method. FIG. 7C is a Venn diagram showing the common and distinct proteins significantly enriched in one fraction as compared to the other two fractions. Significance of differential enrichment was determined by the two-way ANOVA using the area under the curve (AUC) as determined by the label free quantification (LFQ) method, which was prior normalized to SpC. FIG. 7D is a heatmap of the differentially enriched protein in each fraction. FIG. 7E shows non-redundant GO Terms of the differentially enriched proteins in each fraction as determined by Webgestalt analysis. FIGS. 7F-7G show AUC of seven previously identified and novel cell-free transcription factors as quantified in each of the three fractions. Error bars represent SEM of three biological samples.

In one aspect the present method and system comprises a chromatography method based on a gradient of size exclusion multi-bead column which allows one-dimensional sub-population EV isolation denoted herein as Particle Purification Liquid Chromatography (PPLC). Unlike fast purification liquid chromatography (PPLC) and high performance liquid chromatography (HPLC) systems, PPLC is for particles such as EVs, viruses, liposomes and synthetic nanocages. In another aspect, use of the drop-based fraction collector with PPLC allows collection of as little as 22 μl per fraction (1 drop), rendering any sample to be fractionated into as many as ˜3000 distinct fractions, based on the current column parameters where the elution between the void and the total volumes typically spanned over 500 fractions of 6 drops each. The PPLC embodiment of the disclosure can employ a UV-Vis which takes advantage of full UV-Vis spectra in order to (i) accurately identify the fractions, (ii) determine total lipid concentration, (iii) particle size and (iv) particle concentration, as well as (v) assess particle purity, without flow cytometers, NTA, and Tunable Resistive Pulse Sensing (TRPS), or reagents such as antibodies, colorimetric lipid quantification kits which are based on the sulfo-phospho-vanillin colorimetric method.

The PPLC aspect of the present disclosure can detect 47-60 nm particles whereas NTA recorded 118-131 nm. And the turbidity-based calculations of the disclosure do not require dilute samples as opposed to NTA which only operates in a narrow concentration range of very diluted particles, making turbidity, but not NTA, suitable for on-line tandem analytical and preparative systems such as PPLC. The 3D UV-VIS profiles of PPLC as in the disclosure precisely distinguishes EVs from protein aggregates, and NA-rich components unlike NTA. PPLC in the method disclosed can also enrich RNA species of interest during EV isolation. MC-associated RNA can also be queried using PPLC. PPLC can separate biofluids into fractions that contain distinct proteins/peptides, although some overlap was observed. UV-Vis detection in PPLC, unlike light scattering, is compatible with preparative separations, with a dynamic range of detection from micro-absorbance to absorbance units. PPLC as used in the method of the disclosure can isolate, characterize, and retrieve MCs that concentrate a wide array of bioactive molecules without an encapsulating membrane. Indeed, co-purification of EVs and MCs or other contaminants is an undesirable feature of most EV isolation protocols. PPLC solves this problem. In the practice of the method disclosed, EVs from MCs and avoid contaminants that often times confound results in EV studies. Practically, it has been reported that cell-free proteins and nucleic acids co-purify with EVs when other isolation methods, such as miRCURY™ Exosome Isolation Kit were use. Precipitation based EV isolation could co-precipitate lipoprotein, 9-15% of plasma proteins, and 21-99% of vesicle-free miRNAs, as well as depending on the individual miRNA. Therefore, the PPLC solves this problem by separating EVs from MCs and other macromolecules, including all from a single sample tube. Finally, PPLC algorithm and use of UV-Vis/turbidimetry calculations provides real-time understanding of biological processes within biofluids that may allow the physiological status of the producer cells to be monitored continuously.

### Example 2

This Example is to the use of Particle Purification Liquid Chromatography to distinguish HIV-1 from Host Cell Extracellular Vesicles.

The media of U1 cells was chosen, a chronically infected HIV-1 cell line, with a focus to separate HIV particles from host cell extracellular vesicle (EVs). The PPLC method and analysis of the disclosure indicated that HIV-enriched fractions elute prior to the EV-enriched fractions.

HIV-1 self-assembles near and underneath the plasma membranes by forming a dense viral genome-containing capsid covered with an envelope of gag- and gag-pol-polyprotein, and ultimately buds on the outer surface of the membrane engulfed by a tight lipid bilayer. This biogenesis pathway through direct budding resembles that of microvesicles, a class of large EVs, more than that of exosomes, a tetraspanin-rich EV class that are secreted through exocytosis. HIV-1 particles may be closer in size to the microvesicles, thus larger than exosomes, the most abundant class of EVs. The PPLC separation method of the disclosure is used to distinguish infectious particles from exosomes. Furthermore, thin section electron microscopy have shown that immature HIV-1 (132 to 146 nm) are slightly larger than the mature particles (110 to 128 nm), further indicating utility of the high-resolution size-guided PPLC approach of the disclosure to purify HIV.

In this Example, the PPLC method of the disclosure was used to separate HIV from exosome from U1 cell culture supernatant. Results showed that HIV readily elutes earlier than tetraspanin-rich EVs, although with some degree of overlap.

Methods: PPLC efficiently separates HIV-1 particles from EVs from cell culture supernatant:

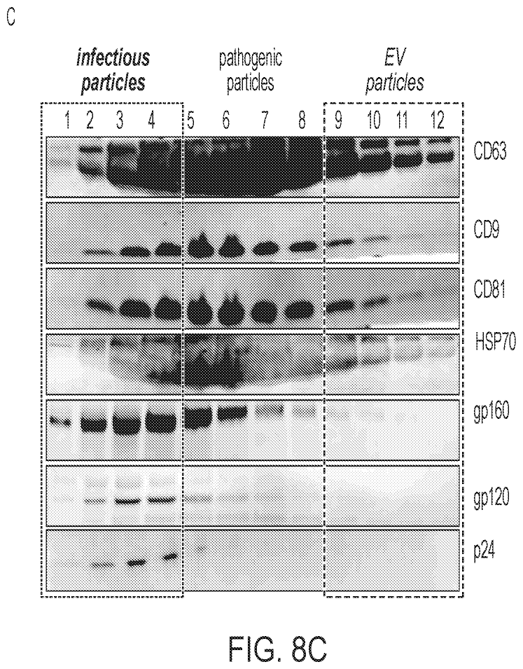

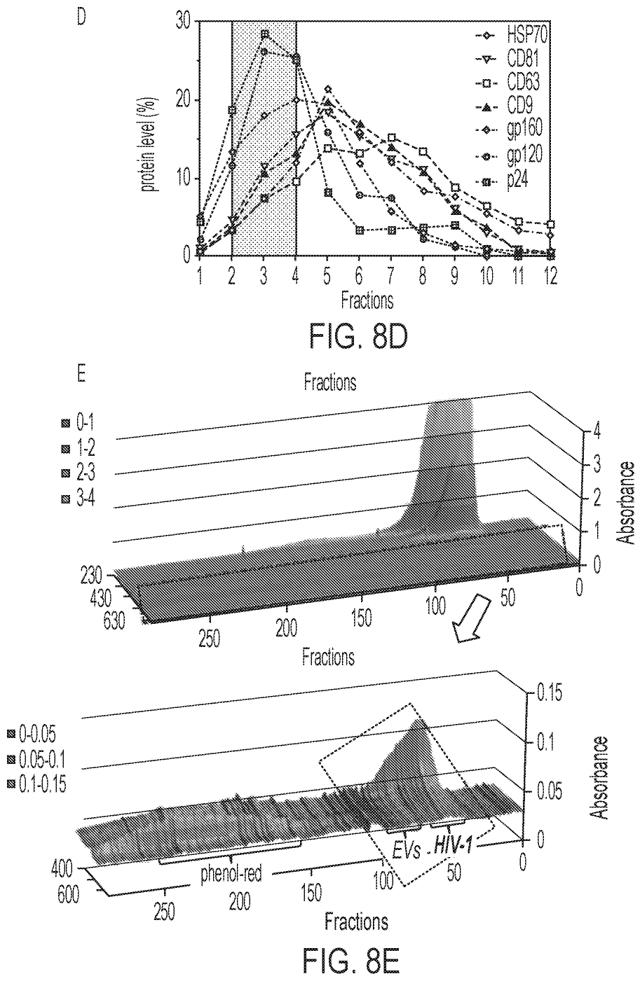

45 million U1 cells, which are U937 monocytes that are chronically infected with HIV, were induced with 50 ng/ml Phorbol 12-myristate 13-acetate (PMA) and cultured at a 3M/ml density in a 150 mm tissue culture dish for three days. The supernatant was collected, concentrated to 1 ml, and loaded onto PPLC. In contrast to seminal and blood plasma which feature four and three PPLC peaks respectively as shown in Example 1, only two peaks were noticeable: a major first peak and small second peak (FIG. 8A). Nonetheless, twelve individual fractions were collected as shown (FIG. 8A, inset) and subjected to protein quantification and acethylcholine esterase (AChE) activity which showed that proteins peaked in F3-8 whereas all fractions had similar AChE activity above baseline (FIG. 8B). Western blot analysis revealed that F1-4 were enriched in HIV-1 proteins p24, gp160 and gp120 with a maximum in F3-4, while CD63, CD9, CD81, and HSP70 spanned to most of the fractions from F2-12. HSP70 peaked at F4-6, while and CD63, CD81, and CD9 peaked at F5-7 (FIG. 1C). ImageJ quantification of the bands indicated enrichment of HIV in the F2-4, whereas the HIV-free vesicles eluted later, Thus, HIV-1 enriched fractions essentially eluted in the ascending part of the first peak and EVs enriched fractions eluted in the descending part of the peak. To evaluate the 3D UV-Vis feature of the PPLC system of this disclosure, full spectra obtained during the separation were plotted (FIG. 8E). The top graph (full spectra) showed high-intensity signal of the first peak in the UV range (230-350 nm). However, a zoom-in into the turbidity range (400-600 nm, bottom graph) showed a deconvolution, albeit low, between two sub-peaks corresponding to the HIV-rich and EV-rich particles, as identified above. Finally, the later fractions (150-250) exhibited peaks at ˜440 and ˜550 nm. Such fingerprint is typical of phenol red and can be quantified from the spectra. Hence, it can be used as a loading control for conditioned-media based separations. The PPLC method of the disclosure can be used as a simple but robust one-step separation of infectious HIV particles from host cell EVs without invoking an ultracentrifugation step. Furthermore, the 3D UV-Vis feature of PPLC can be used to distinguish different particle species, otherwise impossible to recognize in a single-wavelength profile. Ongoing efforts focus on column chemistry optimization to achieve a better separation.

With more specific reference to FIGS. 8A to 8E, which relate to HIV-1 separation from conditioned cell cultures EV by the PPLC method of the disclosure. FIG. 8A 1 ml clarified concentrated supernatant was purified on a 100×1 cm SEC gradient column using PBS as mobile phase and fractions were collected in a 96 well plates, 6 drops/well using a FC204 Gilson fraction collector. Absorbance at 280 nm is shown. Inset is zoom-in of the peak as pointed by an arrow. Fractions highlighted in red were chosen for subsequent analysis. FIG. 8B shows Bradford analysis of the 12 chosen fractions showing a protein concentration apex between F3 and F8. Error bars represent SD of duplicate measurements. Right axis represents AChE activity. Error bars are SD of triplicate measurements. Blue and red dashed horizontal lines represent PBS control, for bradford and AChE, respectively. FIG. 8C is a Western blot analysis of EV markers and viral proteins. 2 microliters of each fraction were mixed with Laemmli buffer and separated on a 4-20% TGX precast gel (Bio-Rad). The proteins were transferred to a PVDF membrane before blocking with 5% BSA and incubating overnight with corresponding primary antibodies. The membrane was washed and incubated for 1 h with a solution of the appropriate fluorescent secondary antibody (Li-COR) before imaging using Li-COR Odyssey imaging system. FIG. 8D is an ImageJ quantification of the bands in FIG. 8C. Levels are reported as a percentage of the total amounts. Gray bar highlights HIV viral particles. FIG. 8E is a 3D UV-Vis profile of the separation, with the turbidity range zoomed-in in the bottom graph as denoted by the red arrow. Dashed rectangle in bottom graph denotes 2 subsequent shoulders, which corresponded to HIV-1 and EVs, respectively. The latter fractions (150-250) showed peaks at ˜440 nm and ˜550 nm indicating the presence of phenol red. Graphs in FIG. 8E were plotted using the Excel application from Microsoft Office 2019.

### Example 3

This example is to the Identification of HIV-1 Inhibition Subpopulations from Seminal Plasma through Particle Purification Liquid Chromatography.

Seminal plasma is a rich biofluid that contains factors related to reproduction and transmission of infective agents. In addition, seminal plasma harbors various antibacterial and antiviral factors, especially anti-HIV-1. However, the distribution of the anti-HIV-1 factors in seminal plasma is unknown. In this example, the Particle Purification Liquid Chromatography (PPLC) method of the disclosure is used to identify the fraction of seminal plasma with HIV inhibition properties. PPLC-purified seminal plasma fractions were then pooled as subpopulations of large and small seminal extracellular vesicles (SEV_{L} and SEV_{S}), and membraneless condensates (MC), and co-incubated at various concentrations with HIV-1 before cellular infection. Results showed that SEV_{L} and MC, but not SEV_{S} exhibited strong HIV inhibition. Furthermore, SEV_{L} and MC inhibited exogenous tat-mediated HIV promoter activation, indicating that the anti-HIV factors in SEV_{L} and MC may target viral transcription step of the HIV lifecycle.

Seminal plasma is a rich biofluid that contain a myriad mixture of immunomodulatory and cytotoxic factors that play crucial roles in reproduction and transmission of sexually transmitted infections (STIs), but also harbors various antibacterial and antiviral factors, especially anti-HIV-1. As known in the art, anti-HIV factors in seminal plasma are particularly enriched in exosomes, a class of extracellular vesicles, thus narrowing the focus towards a particular molecular subset of seminal plasma to isolate the HIV inhibitors.

The PPLC method of the disclosure was used to fractionate human seminal plasma into sub-populations, which were tested for anti-HIV function. Results presented here show that the seminal plasma anti-HIV factors are enriched in SEV_{L}, SEV_{S} and MC, two compositionally distinct subpopulations, although some inhibitory activity is also noticed in SEV_{S} at higher concentrations.

Methods: Anti-HIV subpopulations were identified through PPLC from seminal plasma

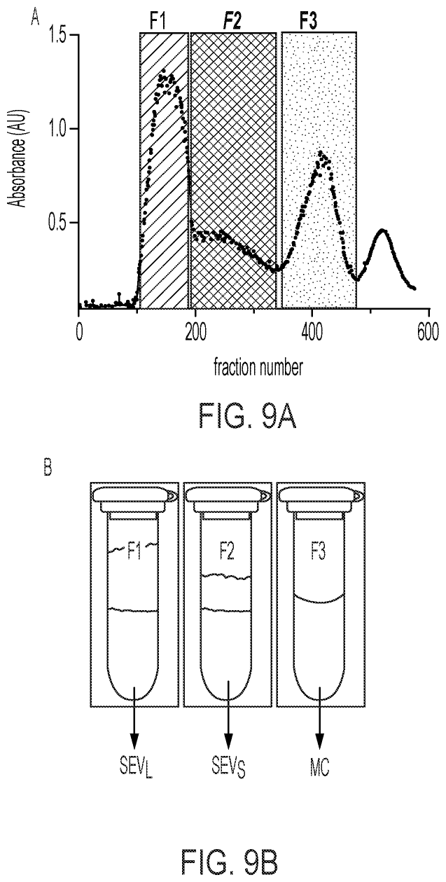

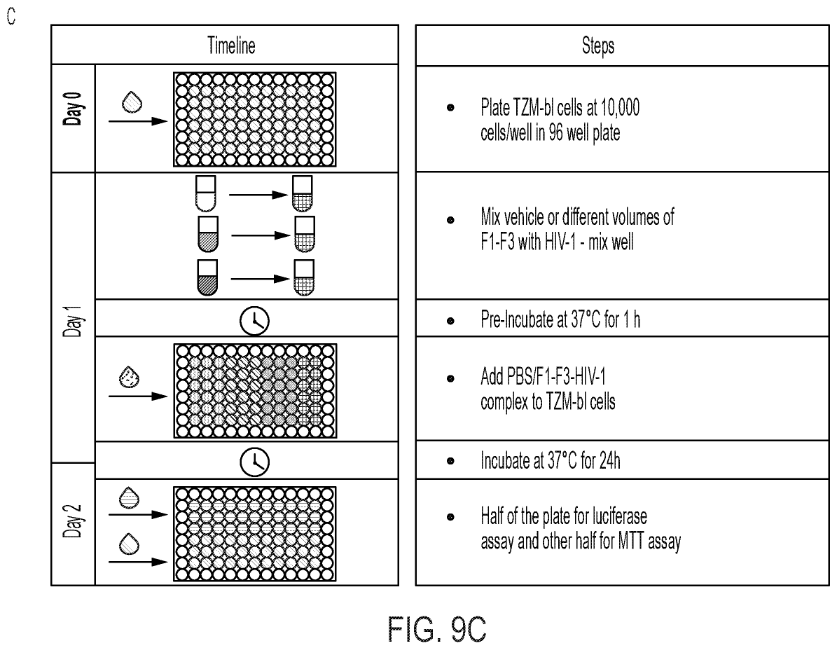

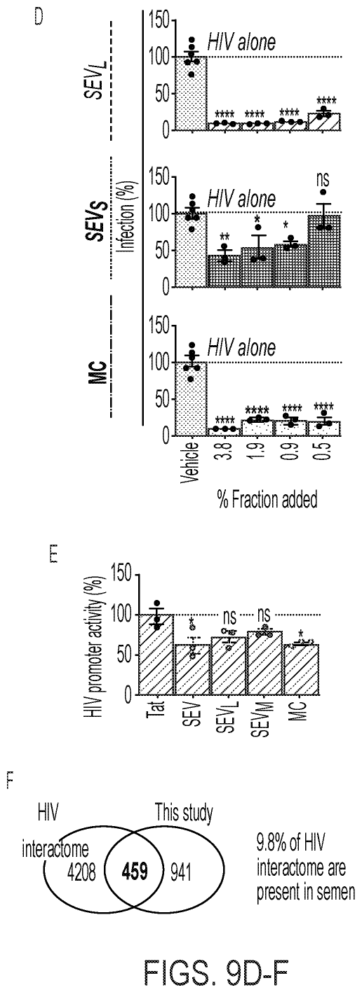

1 ml of seminal plasma was clarified by 2000×g for 10 min and 10,000×g for 30 min before loaded onto the PPLC system. The absorbance profile showed formation of four peaks (F1-F4) as indicated in FIG. 9A. The sample fractions were collected and pooled based on the highlighted regions in FIG. 9A, and the volume of the factions were adjusted to the input volume (FIG. 9B). As indicated in Example 1, F1-F2 comprised membrane encapsulated vesicles, F3 comprises membraneless particles and F4 comprises small molecules that are lipid-free and protein-free, therefore the fractions here were denoted as F1: seminal exosome large vesicles (SEV_{L}), F2: seminal exosome small vesicles (SEV_{S}), F3: membraneless condensate (MC) and F4: small molecules (SM). Since SM fraction lacked protein and lipid content, SEV_{S} and MC were used to test the anti-HIV effect. HIV infection assay is illustrated in FIG. 9C. Briefly, different concentrations of fractions are incubated with pNL4-3 HIV for 1 hour at 37° C. before infection of TZM-bl cells plated the previous day. 24 hours later, HIV infection was assessed by Steady-Glo luciferase assays. Results showed that both SEV_{L} and MC inhibit HIV inhibition and Tat-mediated HIV promoter activation (FIGS. 9D and 9E). Proteomics study also indicated that potential anti-HIV molecules were predominantly present in seminal extracellular particles (FIG. 9F). The PPLC separation method of the disclosure provides for retrieval of intact particles with biologically functional materials. The fact that all three seminal plasma fractions exhibited some levels HIV inhibitory activity suggests that the anti-HIV factors are prevalent in seminal plasma.

With more specific reference to FIGS. 9A to 9F, which relate to the identification of anti-HIV factor through PPLC from seminal plasma. FIG. 9A 1 ml clarified seminal plasma was loaded onto a 100×1 cm SEC gradient column using 1×DPBS as mobile phase. Fractions were collected in a 96 well plates, 6 drops/well using a FC204 Gilson fraction collector. Absorbance at 280 nm is shown. Fractions highlighted in three colors were chosen for subsequent analysis. FIG. 9B shows the collected fractions were adjusted to the same volume as the loaded volume. FIG. 9C shows a schematic method of an infection and viability assay. FIG. 9D shows the effect of HIV infection of Tzm-bl with SEV_{L}, SEV_{S} and MC treatment. FIG. 9E shows the effect of Tat-mediated HIV promoter activation on Tzm-bl with SEV_{L}, SEV_{S} and MC treatment. FIG. 9F is a Venn diagram showing the presence of 459 known HIV-interacting proteins in seminal plasma analyzed in the present study.

### Example 4

This Example is to the use of PPLC for purification of blood extracellular vesicles from albumin and other impurities.

In this Example isolates albumin-free EVs from blood plasma, which heretofore has been difficult to accomplish. Blood plasma EV preparations are often contaminated with albumin hindering blood EV-based biomarker discovery. The results presented here demonstrate that PPLC in the method of the disclosure can readily be applied for EV isolation from blood plasma although some EV/albumin overlap is still noticeable. Of note, the SEC gradient column used in this Example was for seminal plasma EV-MC separation. Blood plasma is the most studied body fluid for physiological and pathological assessments during routine clinical checkup and hospitalization, but also in diagnostics. Fetal medicine presents one example of early diagnostic tests such as maternal blood screening for fetal genetic disorders; i.e., trisomies 21, 18 and 13 in pregnancy, fetal aneuploidies, Down, Edwards and Patau syndromes, but also for very early fetal sex determination. Liquid biopsy is another popular example of blood plasma utility in various cancer diagnosis and prognosis such as lymphomas, rectal, ovarian, breast, and pancreatic cancers, to cite but a few. The development of these precision medicine tests rely on the detection of unique biomarkers, whose levels define the medical state of the patient and predict the disease progression. Examples of blood plasma proposed biomarkers include circulating miRNAs cell free DNA (cfDNA), proteins, metals, and more recently extracellular vesicles. These approaches have been limited by assay sensitivity. Indeed, outdated methods of blood plasma processing for protein, DNA, or RNA isolation are still being employed in clinical and research studies.

Albumin is the most abundant circulating protein in the blood plasma and its presence poses challenges for proteinaceous biomarker discovery by proteomics. Albumin depletion protocols have been proposed. However, current protocols are problematic because they often employ organic solvents which denature the proteome a detrimental consequence for any biological relevance. Immunoprecipitation technique were also used, but they are known for their low specificity in proteomics studies. Plasma delipidation is another protein purification method but it risks removing membrane-associated proteins such as those present in EVs. Furthermore, it has been shown that albumin depletion also removes low abundance biomarkers including cytokines and albuminome analysis revealed that critical plasma proteins are actually shuttled with albumin. There is a need for development of a purification method that, instead of depleting plasma components such as albumin, rather fractionates plasma into different component-rich fractions, in non-denaturing minimal shear stress settings to preserve the inherent concentrations and interactions in the plasma. This Example employed a PPLC method of the disclosure to fractionate blood plasma without eliminating any plasma component. The results show that blood plasma can be readily fractionated into more than 500 size-guided fractions. Furthermore, the results show for the first time direct detection of EV markers by western blot, without the need of any blood plasma component removal. Western blot quantification shows 85% removal of albumin from EV-enriched fractions. The results also show that chromatogram profiles obtained by PPLC can accurately predict EV-containing fractions from blood plasma.

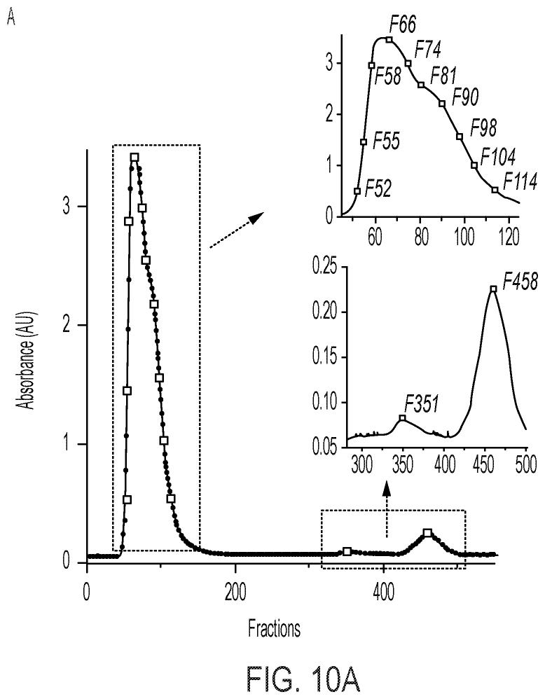

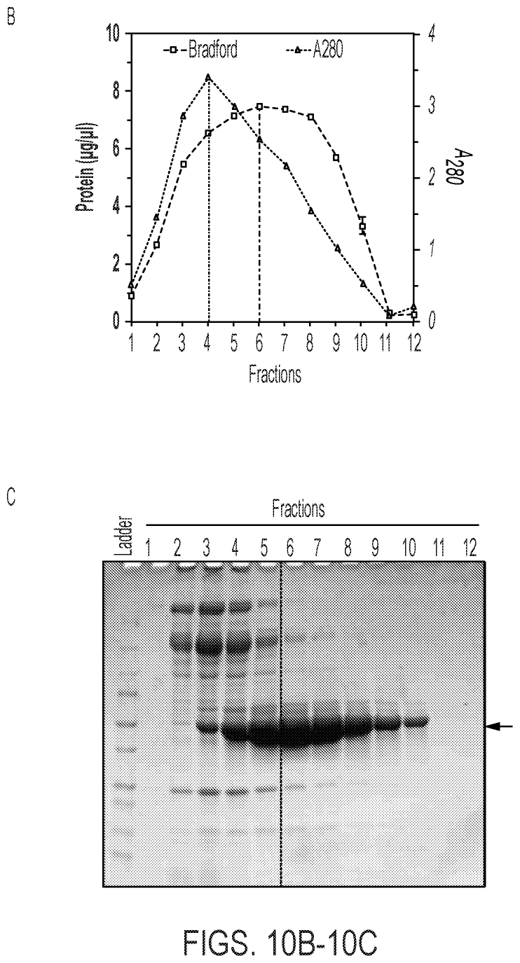

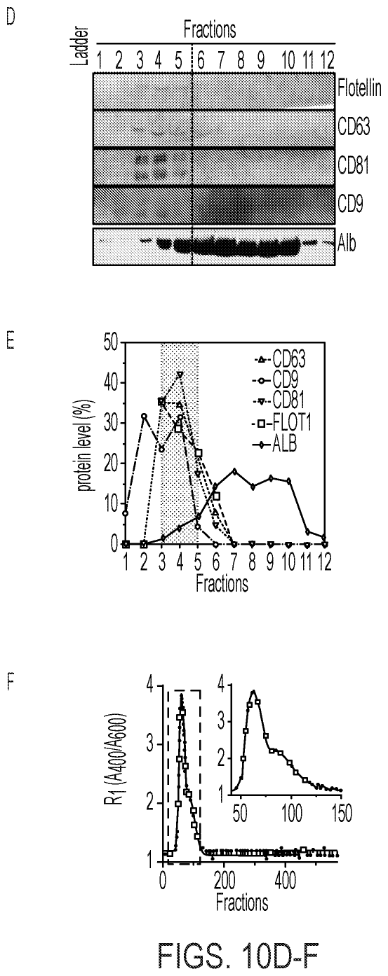

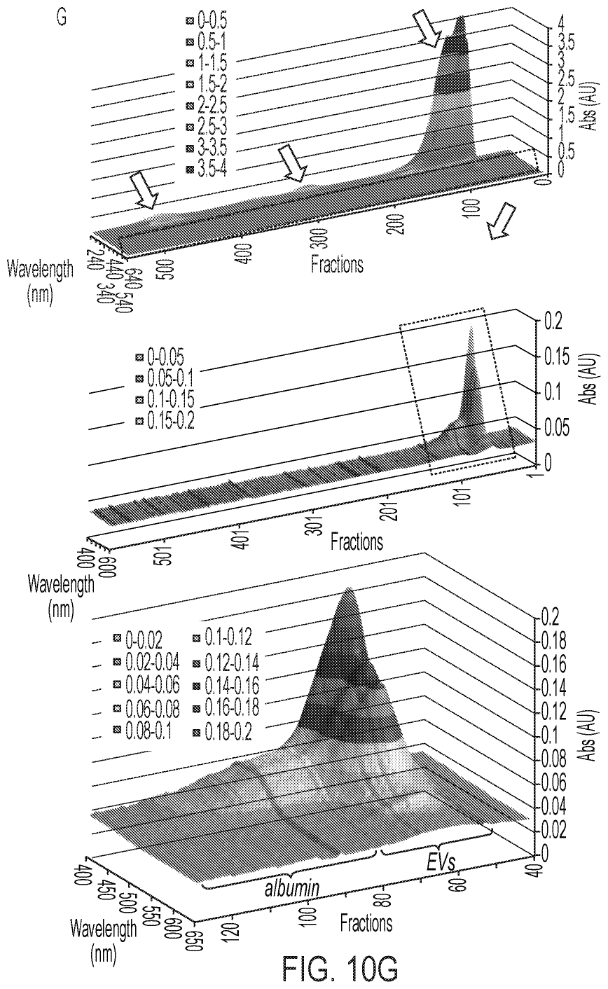

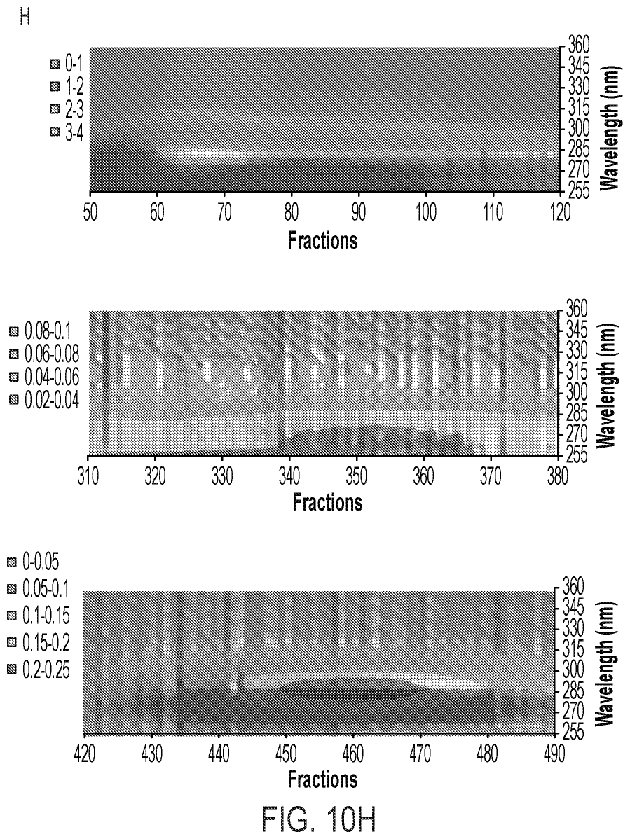

Methods: 1 ml of a 4-donor pool of clarified blood plasma was purified by size-exclusion gradient PPLC system. Absorbance at 280 nm (A₂₈₀) showed that most of the plasma components eluted in a first large peak and 2 other small peaks (FIG. 10A). Twelve individual fractions from these peaks were collected as shown. The fractions were subjected to protein quantification by Bradford assay, which revealed that, despite A₂₈₀ peaked at F4, F6 contained the highest protein amount (FIG. 10B). SDS-PAGE analysis showed that immunoglobulin-size proteins notably eluted in the early fractions whereas albumin-size bands peaked at F5 (FIG. 10C, black arrow). Western blot analysis revealed that F2-4 were enriched in major EV markes—CD63, CD9, CD81, FLOT1, while F5-10 were mostly enriched in ALB (FIG. 10D). Band intensity quantification of the different markers showed that F3-5 contained most of the EVs with only ˜15% albumin. The rest of the albumin were within F6-F10 (FIG. 10E). Thus, EVs essentially eluted in the ascending part of the first peak while albumin eluted relatively later in the descending part of the first peak. Interestingly, this analysis correlated to the turbidity (r) index, which showed within the first peak a first sharp peak and a second short tailing peak (FIGS. 10F and 10F inset). Detailed analysis of the 3D UV-Vis profile showed that these two sub-peaks in peak 1 (F50-120) exhibited different spectra in the turbidity range (400-600 nm), pointing to the enrichment of the former in EVs whereas the latter was albumin-rich (FIG. 10G). In contrast, The UV range (255-360 nm) showed no difference within the first peak where all fractions peaked at 280 nm (FIG. 10H, top). However, the UV range profiles showed different peaks 2 (F310-380) and 3 (F420-490), in which peak 2 and 3 exhibited a maxima of ˜260 nm and ˜285 nm, respectively; suggesting the presence of detectable, albeit low, cfDNA, in peak 2 and short peptides/free amino acids in peak 3. In summary, these analyses clearly demonstrate the applicability of PPLC and its ability to efficiently separate albumin from EVs in a one-step protocol.

With more specific reference to FIGS. 10A to 10H, which relate to the efficient removal of albumin from blood plasma preparation during EV isolation. For FIG. 10A, 1 ml clarified blood plasma (n=4) was purified on a 100×1 cm gradient column using PBS as mobile phase and fractions were collected in a 96 well plates, 6 drops/well using a FC204 Gilson fraction collector. Absorbance at 280 nm is shown. Fractions highlighted in red were chosen for subsequent analysis. FIG. 10B shows Bradford analysis of the twelve chosen fractions with a protein concentration apex at F6, whereas A₂₈₀ absorbance apex was at F4. Error bar are SD of duplicate measurements. FIG. 10C shows SDS-PAGE of 12 chosen fractions. 2 microliters of each fraction were mixed with Laemmli buffer and separated on a 4-20% TGX precast gel (Bio-Rad) before staining with Coomassie Blue. Left lane correspond to the Precision Plus Protein Standards ladder (Bio-Rad). FIG. 10D shows Western Blot analysis of EV markers and albumin. 10 μg protein from each fraction were separated on a 4-20% TGX gel and the proteins were transferred to a PVDF membrane before blocking with 5% BSA and incubating overnight with corresponding primary antibodies. The membrane was washed and incubated for 1 h with a solution of the appropriate fluorescent secondary antibody (Li-COR) before imaging using Li-COR Odyssey imaging system. FIG. 10E shows ImageJ quantification of the bands in 10D. Levels are reported as a percentage of the total level. FIG. 10F shows turbidity ratio R₁ of the separation profile highlighting a pronounced second peak in the descending fractions. FIG. 10G illustrates 3D-UV-Vis profile with drastic changes in the turbidity region (red arrows) indicating the presence of 2 distinct populations, which corresponded to EV and albumin, respectively. Dashed red rectangles highlight the spectra zoomed-in in the bottom graph. Black arrows indicate the UV region of the spectra that are shown in H. FIG. 10H illustrates a contour view of the UV region highlighted by black arrows in G and shows no apparent differences in the first peak, but detection of a ˜260 nm peak around F350 and a ˜285 nm peak at F460. Graphs in G and H were plotted using the Excel application from Microsoft Office 2019.

The PPLC separation method of the disclosure separates plasma EVs from albumin allowing direct western blot detection of EV markers using a one-step protocol without invoking an ultracentrifugation step. The PPLC-based separation disclosure can be used to enrich for i) blood EVs devoid of most albumin, and ii) albumin fractions devoid of most EV proteins in non-denaturing conditions, thus preserving the native structures of the components of interest.

What is claimed is:

1. A method for isolating intact acellular particles comprising:
(i) providing a biofluid sample containing intact acellular particles of different sizes;
(ii) separating the biofluid sample using a size exclusion gradient into subpopulations of intact acellular particles, each respective subpopulation individually comprising a different size range of intact acellular particles; and
(iii) isolating a respective subpopulation.

2. The method of claim 1 wherein the biofluid sample comprises one or more of the following: a biological fluid, a body fluid, a culture fluid obtained from a human cell, a culture fluid obtained from a bacterial cell, a culture fluid obtained from a fungus cell.

3. The method of claim 1 further comprising:
(iv) analyzing the respective subpopulation to determine size of the intact acellular particles therein, the concentration of the intact acellular particles therein, or both.

4. The method of claim 1 wherein the intact acellular particles comprise extracellular vesicles, membraneless condensate particles, or both.

5. The method of claim 1 wherein the separating step of (ii) comprises contacting the biofluid sample with size exclusion beads to separate the intact acellular particles into the subpopulations.

6. The method of claim 5 wherein the size exclusion beads comprise different pore sizes and are configured to form the gradient going from largest pore size to smallest pore size and wherein the biofluid sample progressively contacts the gradient from largest pore size to the smallest pore size.

7. The method of claim 1 wherein the isolating step of (iii) comprises collecting the respective subpopulation as a fraction of the biofluid using a fraction collector.

8. The method of claim 3 wherein the analyzing step (iv) comprises obtaining light scattering information for the respective subpopulation, and wherein the light scattering information is used to determine the size and the concentration of the intact acellular particles contained in the respective subpopulation.

9. The method of claim 8 wherein the light scattering information includes absorbance by the respective subpopulation of light in the visible range of between about 400 nm to about 600 nm.

10. A method for isolating intact acellular particles comprising:
(i) providing a biofluid sample containing intact acellular particles of different sizes;
(ii) feeding the biofluid sample to the inlet of a particle purification liquid chromatography column comprising size exclusion beads having different pore sizes, the size exclusion beads layered within the column to provide a gradient along the length of the column wherein the largest pore size is at the inlet of the column and the smallest pore size is at the outlet the column, the biofluid sample flowing through the column from the inlet to the outlet to progressively elute respective subpopulations of intact acellular particles, each respective subpopulation individually comprising a different size range of intact acellular particles;
(iii) isolating each eluted respective subpopulation into one or more wells of a fraction collector;
(iv) performing UV-VIS spectrometry on the respective subpopulation within the one or more wells to obtain light scattering information for that respective subpopulation and determining from the light scattering information the size of the intact acellular particles contained in that respective subpopulation, the concentration of the intact acellular particles contained in that respective subpopulation, or both.

11. The method of claim 10 wherein the biofluid sample comprises one or more of the following: a biological fluid, a body fluid, a culture fluid obtained from a human cell, a culture fluid obtained from a bacterial cell, a culture fluid obtained from a fungus cell.

12. The method of claim 10 wherein the intact acellular particles comprise extracellular vesicles, membraneless condensate particles, or both.

13. The method of claim 10 wherein the light scattering information includes absorbance by the respective subpopulation of light in the visible range of between about 400 nm to about 600 nm.

14. A system for isolating intact acellular particles comprising:
a station for separating, using a size exclusion gradient, a biofluid sample containing intact acellular particles of different sizes into subpopulations of intact acellular particles, each respective subpopulation individually comprising a different size range of intact acellular particles; and
a station for isolating a respective subpopulation.

15. The system of claim 14 further comprising a station for analyzing the respective subpopulation to determine size of the intact acellular particles therein, the concentration of the intact acellular particles therein, or both.

16. The system of claim 14 wherein the station for separating comprises a particle purification liquid chromatography column comprising size exclusion beads having different pore sizes, the size exclusion beads layered within the column to provide a gradient along the length of the column wherein the largest pore size is at the inlet of the column and the smallest pore size is at the outlet the column, the biofluid sample flowing through the column from the inlet to the outlet.

17. The system of claim 14 wherein the station for isolating comprises a fraction collector.

18. The system of claim 15 wherein the station for analyzing comprises a UV-VIS spectrometer to obtain light scattering information, including absorbance by the respective subpopulation of light in the visible range of between about 400 nm to about 600 nm.

19. An assembly for isolating intact acellular particles comprising, in combination:
a particle purification liquid chromatography column having an inlet and an outlet and configured to flow therethrough a biofluid sample containing intact acellular particles of different sizes, the column comprising size exclusion beads having different pore sizes, the size exclusion beads layered within the column to provide a gradient along the length of the column wherein the largest pore size is at the inlet of the column and the smallest pore size is at the outlet the column;
a fraction collector configured to receive an elute from the outlet; and
a UV-VIS spectrometer configured to obtain light scattering information on the elute.

20. The assembly of claim 19 further comprising, in combination: an analyzer to determine the size of an intact acellular particle in the eluate, the concentration of an intact acellular particle in the eluate, the refractive index of an intact acellular particle in the eluate, or all together.

21. The method of claim 3 wherein the analyzing step comprises identifying in the respective subpopulation cell-free nucleic acids, anti-HIV factors, or both.
